SEQUENCE LISTING (31 sequences)

SEQ ID NO: 1 - DNA, 2172 bp, Mus musculus
agatcttacttcaagttccgggaatatccaggtgtagcttttcttttcaaaactccctggtccgttagtcagatcccctgagccataacttccaccgctccccaataactctcctgtagcatcccctaaaatggagaggtgagagccagaatccgagttacatttcactgaatgaggatatgccaactcttcatagaagggctgtcccaataaaatctgcctctactgtgcttaaccttcaagatttttctgcggatccaggtatttaaacgcaacacccagagtaaatgaaaaaaaacaaaaacaaaacaaaaaaacaacccccccccccaaaaaaaaagacaatgctccttgcttgctctgctctcagccagttgacagaacaaagaaaaagaaaaactccaggcagtttatctctagaatcccagaatgattggtgccatgttttaggccaccaaagaactcccacaatgccttgcttttctttttagattcgttccgggtctggaaggttactttcaatatcttgcaaatgcccaggatctccttagaaccctctccccctcccctttcccagatgtaccccattccagccactcaaccctgacactgtttgcaaactcaagggcaattgcctttacataagcaattcaagtaggatgcattcattgcaaaggggttgaagagggtaagaacttaatgaagagttagaagaggactggaaagagaaggaggccaccattcctcctcctcctctgtcacttttcttgggctagaaggacggaggatcatccatctgaaagattcctccttcagatggagaaaataaagactccttatttaagaatagataaggctattccacaatagactttgcttgactcaaaacacagagtccccagaaagggaaacagcacattgaatagagttttattgcagcttctctttgagcttcttctgaccaaggccccagagattgagcactaggggctcaaggaaggctgtgattgaattatggtctgtgtcagagatgaaattcccacctctggtgcggggttgtttaatcttagcattccctagatgagctagctgaggtcattcattactccaggagcatctgaaaaccaacggagccaaggtctgctggcaaaaactagctttagacacaaaattctagggtttagagttgaagactcttagtgaagtctctggggtaggagcaggtgaccgttaggtttacatttggagtgttcaagtgtagggtatggagctgacttgcaaaggattctagaagatgctctagtcaggaaggttgagtcaaggctgtgtttccagatgaggtcgtttttatatgaaataaatgtttgcctagagaggtgatggagattagaagccagccaccctttggtggcaataagcaaagcctctgctaagaggtagaagctaagagatgggcctctccagagagaacaaagtcaaactgcaccaccggagaccattgtcaagggaaggacccttcagtgcacctcagtatctgggattgtgagtggttcacctctgtctacctctctgcgctaaagcagtcttcaggctaggacatagactcagtgatctttctttacctttccagctcgcctcccgcaaccccccccccccccacatgtcctctgtcttctgctgccacaaagggttaatctctcctgcgggtaaaaacaggtccgcggagtctctaactggcgacagatgggccactttcttctggccacaaaggggccggaatggagcgctccgcggcatacaaataggcaggtcacgtggttcccggctcttggctggaccgggaagaccatatggcgcatgccggggaggaaggagcaggggcggaggtactgtgggggtgaggggagtggtggtggaagggggcgggaggaaagttctggggaggggtgaatgagggcgggagtcgttcagtctggacgcgtgcgcgattgcggggcgcagggacctgctcgttgctcagctcacggccccggcccgcgctcagcatcagcaactcggctatataaccctagcgcccgcgccaccgggacacgaggaattcgcccacgcagaaggcaaggtgtcccgaggctccagggttatgagatcgtcactattcagaaccttttaaca

SEQ ID NO: 2 - DNA, 7 bp, Artificial sequence
  consensus nucleotide sequence
wwcaawg

SEQ ID NO: 3 - DNA, 22 bp, Artificial sequence
  oligonucleotide primer
aagagagaggccctcagttgct

SEQ ID NO: 4 - DNA, 22 bp, Artificial sequence
  oligonucleotide primer
ttgtgagggagatgctcagtgt

SEQ ID NO: 5 - DNA, 26 bp, Artificial sequence
  oligonucleotide primer
gaagaacagcccggaccgcgtcaaga

SEQ ID NO: 6 - DNA, 27 bp, Artificial sequence
  oligonucleotide primer
cttcatgtgcagagcgcgcagccgctt

SEQ ID NO: 7 - DNA, 25 bp, Artificial sequence
  oligonucleotide primer
gcaccatggacagctcccatgtctt

SEQ ID NO: 8 - DNA, 25 bp, Artificial sequence
  oligonucleotide primer
cctctaatcgtgaaagatggcatta

SEQ ID NO: 9 - DNA, 26 bp, Artificial sequence
  oligonucleotide primer
gacgacgacgagccgaggagcgacaa

SEQ ID NO: 10 - DNA, 25 bp, Artificial sequence
  oligonucleotide primer
cgatcttggacagcttctgcgtctt

SEQ ID NO: 11 - DNA, 25 bp, Artificial sequence
  oligonucleotide primer
cctcctactccagttactctggcta

SEQ ID NO: 12 - DNA, 26 bp, Artificial sequence
  oligonucleotide primer
cgtgatgggataaacaggctgacctt

SEQ ID NO: 13 - DNA, 26 bp, Artificial sequence
  oligonucleotide primer
gactccctatgaccacatgactagca

SEQ ID NO: 14 - DNA, 27 bp, Artificial sequence
  oligonucleotide primer
ctgttgtagtcggcagtgctggctgaa

SEQ ID NO: 15 - DNA, 26 bp, Artificial sequence
  oligonucleotide primer
gctcagagggtccgagctgccatgtt

SEQ ID NO: 16 - DNA, 25 bp, Artificial sequence
  oligonucleotide primer
caagcattttcaccagcgctgggat

SEQ ID NO: 17 - DNA, 19 bp, Artificial sequence
  consensus nucleotide sequence
nnagcaccnnggacagnnn

SEQ ID NO: 18 - DNA, 28 bp, Artificial sequence
  oligonucleotide primer
caaggctgtgtttccagatgaggtcgtt

SEQ ID NO: 19 - DNA, 28 bp, Artificial sequence
  oligonucleotide primer
ctttgtggcagcagaagacagaggacat

SEQ ID NO: 20 - DNA, 27 bp, Artificial sequence
  oligonucleotide primer
ccagcaagcagccctataaagcaagca

SEQ ID NO: 21 - DNA, 27 bp, Artificial sequence
  oligonucleotide primer
ctactctgttcacctggttttcatctt

SEQ ID NO: 22 - DNA, 24 bp, Artificial sequence
  oligonucleotide primer
atgtctatttagaatcggcaccat

SEQ ID NO: 23 - DNA, 25 bp, Artificial sequence
  oligonucleotide primer
gtcatcatctttactatatgcaagc

SEQ ID NO: 24 - DNA, 24 bp, Artificial sequence
  oligonucleotide primer
gttcacagttccatggcgtccttg

SEQ ID NO: 25 - DNA, 25 bp, Artificial sequence
  oligonucleotide primer
gcaggttttggttagccattcatgt

SEQ ID NO: 26 - DNA, 24 bp, Artificial sequence
  oligonucleotide primer
ctctcaccccttttatgttgagca

SEQ ID NO: 27 - DNA, 25 bp, Artificial sequence
  oligonucleotide primer
cttcattacatgagcatcatgagcc

SEQ ID NO: 28 - DNA, 27 bp, Artificial sequence
  oligonucleotide primer
ccaggactacagcggcagctcagctaa

SEQ ID NO: 29 - DNA, 27 bp, Artificial sequence
  oligonucleotide primer
gtcactgtgaacaaagattagcaccaa

SEQ ID NO: 30 - DNA, 24 bp, Artificial sequence
  oligonucleotide primer
ctctcaccccttttatgttgagca

SEQ ID NO: 31 - DNA, 25 bp, Artificial sequence
  oligonucleotide primer
cttcattacatgagcatcatgagcc

(19) United States
(12) Patent Application Publication
(10) Pub. No.: US 2006/0234378 A1
(43) Pub. Date: Oct. 19, 2006

(54) Molecular switch regulating neurogenesis

(75) Inventors: Tomoko Kuwabara, Ibaraki (JP); Fred H. Gage, La Jolla, CA (US)

Correspondence Address: KLARQUIST SPARKMAN, LLP, 121 S.W. SALMON STREET, SUITE 1600, PORTLAND, OR, 97204, US

(73) Assignee: The Salk Institute For Biological Studies

(21) Appl. No.: 11/400,473

(22) Filed: Apr. 6, 2006

Related U.S. Application Data
(60) Provisional application No. 60/669,660, filed on Apr. 7, 2005.

(51) Int. Cl. C12N 5/08; C07H 21/04; C12N 15/09

(52) U.S. Cl. 435/368; 435/455; 536/23.2

(57) ABSTRACT

The present disclosure relates to a novel transcription regulatory master switch involved in neural development. The disclosure provides compositions and methods for regulating neural lineage specific genes and modulating the differentiation of stem cells into neural lineage cells.

### CROSS REFERENCE TO RELATED APPLICATION

This application claims benefit of U.S. provisional application No. 60/669,660, filed Apr. 7, 2005, the specification of which is incorporated herein in its entirety for all purposes.

### ACKNOWLEDGMENT OF GOVERNMENT SUPPORT

This invention was made with United States government support, pursuant to grant AG008514-17 and AG020938-03, from the National Institutes of Health; the United States government has certain rights in the invention.

### FIELD

The present disclosure relates to the field of molecular and developmental biology. More specifically, this disclosure relates to methods of modulating gene expression and influencing cellular differentiation.

### BACKGROUND

Neuroblasts, the first stage in the differentiation of neural stem cells of toward neurons, are generated from self-renewing neural stem cells. The expression of genes that initiate differentiation of these stem cells into neurons is strongly repressed in neuronal stem cells. The mechanism by which neurogenic genes are repressed in neural stem cells and activated during neuronal differentiation is not well known.

Early in neurogenesis stem cells exit the cell cycle and begin to express immature but committed neuronal genes. Embryonic developmental studies have shown that proneural bHLH transcription factors regulate neurogenesis by directing the exit of neural stem cells from the cell cycle and promoting the expression of neuron-specific genes (Ma et al., Cell, 87:43-52, 1996; Li et al., Nucleic Acids Res., 26:5182-5189, 1998; Guillemot et al., Exp. Cell Res., 253:357-364, 1999; Morrow et al., Development, 126:23-36, 1999; Farah et al., Development, 127:693-702, 2000; Scardigli et al., Neuron, 31:203-217, 2001). Genes of the Neurogenin and NeuroD families appear to be expressed at early stages of neurogenesis in the developing brain (Ma et al., Cell, 87:43-52, 1996; Li et al., Nucleic Acids Res., 26:5182-5189, 1998; Guillemot et al., Exp. Cell Res., 253:357-364, 1999; Morrow et al., Development, 126:23-36, 1999; Farah et al., Development, 127:693-702, 2000; Scardigli et al., Neuron, 31:203-217, 2001; Hirabayashi et al., Development, 131:2791-2801, 2004). For example, Neurogenin 1 and Neurogenin 3 promote neurogenesis by activating NeuroD genes (NeuroD1, NeuroD2 and NeuroD3) during development (Ma et al., Cell, 87:43-52, 1996; Sun et al., Cell, 104:365-376, 2001; Heremans et al., J. Cell Biol., 159:303-12, 2002; Andermann et al., Dev. Biol., 251:45-58, 2002).

Previous efforts have not revealed the regulatory mechanism that controls development of a neural phenotype. For instance, it has not been known whether a master bHLH gene exists that controls neurogenesis and, if so, how such a gene might be regulated to effect a developmental switch between undifferentiated stem cells and committed neural lineage cells. The present invention elucidates the regulatory mechanism that functions as a master switch in neural development, and provides useful compositions and methods for influencing cell fate and regulating neural development, as well as other benefits that are disclosed herein.

### SUMMARY

The present disclosure describes a transcriptional regulatory “master switch” that determines whether the genetic program that mediates neural differentiation is repressed or activated. Based on the identification of response elements, designated LEF/Sox overlapping response elements, within the upstream regulatory sequences of neural specific genes, including NeuroD1, this disclosure describes methods and compositions for directing neural specific expression of polynucleotide sequences and for modulating differentiation of stem cells into neural lineage cells. Additionally, methods for identifying agents that modulate neural differentiation and neural specific gene expression are described.

The foregoing and other features and advantages will become more apparent from the following description of several embodiments, which proceeds with reference to the accompanying figures.

### BRIEF DESCRIPTION OF THE DRAWINGS

FIGS. 1A-B. Expression of genes that regulate the transcriptional machinery of NeuroD1 during neurogenesis. A) Comparison of expression levels between neural progenitor cells (Prog) and cells differentiating to neuronal lineage (neuron) by quantitative RT-PCR of genes controlling expression of NeuroD genes. B) Schematic representation of the binding sites of TCF/LEF and Sox transcription factors on 3-kb promoters of the NeuroD1 and NeuroD2 genes.

FIGS. 2A-C. Transcriptional regulation of NeuroD genes by both TCF/LEF and Sox2 protein. A) The effects of over-expression of transcriptional regulators on the NeuroD1 promoter-driven luciferase gene. B) Analysis of the repression state of NeuroD genes at neural stem cell stage. RT PCR analysis was performed with specific primers for NeuroD1 and NeuroD2 genes after TSA and 5AazaC treatments to see the role of HDACs and DNA methylation on the repression of NeuroD genes. C)CHIP assay for chromatin regulating factors on the promoter regions of NeuroD1 and NeuroD2 genes. PCR primers were designed to surround the DNA elements that have overlapping binding sequences for both Sox2 and TCF/LEF transcription factors. Prog; progenitor.

FIGS. 3A-B. Association of Sox2- and TCF/LEF-transcription factors on the overlapping DNA regulatory element. A) EMSA of Sox2 and LEF1 protein against Sox2, LEF/Sox and LEF1 dsDNA. While the concentration of each nucleotide was fixed as 20 μM, protein amount was increased 2-fold by each lane depending on arrow direction. B) Bar graphs illustrating a representative reporter assay of LEF/Sox overlapping response element on the expression control between repression and activation.

FIGS. 4A-C. Sox2-dependent suppression and β-catenin-dependent activation of NeuroD expression during neurogenesis. A) The effect of over-expression of Sox2-VP16 and of Sox2-Eng on the expression of endogenous NeuroD genes both in neural stem cells (FGF2) and in cells at during neurogenesis (RA+FSK). B) Interactions among transcriptional factors that regulate NeuroD expression during neurogenesis. Proteins that had bound to each specific antibody were “pulled down” and analyzed by Western Blotting. C) Reduction in the up-regulation of NeuroD1 and NeuroD2 genes by β-catenin RNAi. RT-PCR analysis was performed using RNAs extracted from cells infected with RNAi for β-catenin and from cells infected with control lentivirus. D) Schematic representation of Sox2-mediated repression in neural stem cells (left) and β-catenin-mediated activation events in neurogenerating cells (right) through the overlapping DNA regulatory element of Sox2-(green) and TCF/LEF-transcriptional factors (red) on NeuroD promoter.

FIGS. 5A-D. Expression of NeuroD1, LINE1 and NRSE smRNA at early neurogenesis stage in adult hippocampal neural stem cells. A) Time course of Northern blotting of NRSE smRNAs. RNAs from cells treated with 1 μM retinoic acid (RA) and 5 μM forskolin (FSK) for 1-7 days were subjected to see the expression levels of NRSE smRNAs. B) Expression levels of NeuroD1 and LINE1 in RT-PCR analysis during neuronal differentiation. C) Expression levels of β-catenin in RT-PCR and Western blotting analysis during neuronal differentiation. D) The effects of genes contributing to the regulation of the NeuroD1 gene on the expression LINE1 and NRSE smRNAs. Total RNAs from cells that over-express Sox2, HDAC1, CtBP1, Wnt3 and constitutively active β-catenin in adult neural stem cells were used for RT-PCR and Northern blotting analysis.

FIGS. 6A-B. Regulatory elements shared in the transcriptional machinery of NeuroD1, LINE and NRSE smRNA. A) Schematic representation of the binding sites of TCF/LEF and Sox transcription factors on 3-kb promoters of the NeuroD1 gene (within dashed box) and on human, rat and mouse retrotransposon LINE1 genes. Each shaded box represents an overlapping DNA regulatory element for both Sox and TCF/LEF. Since the information of UTR sequences of rat LINE1 is not known, the region was indicated by a dotted line. B) The promoter activity of LINEs during adult neurogenesis. The NeuroD1 promoter, 5′ UTR regions of human and mouse LINE, mouse LINE1 ORF2, mouse truncated LINE, and partial ORF2 from human, mouse and rat LINE1 were cloned and linked to the luciferase gene. Each luciferase construct was introduced into adult hippocampal neural stem cells by electroporation and was normalized by Renilla luciferase construct as an internal control.

FIG. 7. Genomic distribution of NRSE sequence and retrotransposon LINE in mouse genome. Most chromosomes contained adjacent NRSE-LINEs sequences, except for the Y chromosome.

FIGS. 8A-B. Stage-specific up-regulation of genomic transcriptions of nearby NRSE sequence from embedded LINEs inherent promoter. A) RT-PCR analysis for RNA with NRSE sequences surrounded by multiple LINEs on both sides (LINE-NRSE-LINE). Specific primers for reverse transcription were used to hybridize with only sense or antisense RNA transcripts on the locus. B) Northern blotting analysis for LINE-NRSE-LINE RNAs. The probe was designed to hybridize to the flanking region between LINE and NRSE on LINE-NRSE-LINE RNAs (outlined). Prog., RNA from neural progenitor cells; Neuron, RNA from cells treated with RA+FSK for 2 days.

FIGS. 9A-E. Neuronal differentiation by the NRSE smRNA generated from the precursor dsRNA. A) Northern blotting analysis for precursor RNAs. The accumulated NRSE smRNAs were detected when both precursor strands were forced to be expressed in neural stem cells under FGF2. B) Up-regulation of NRSE containing neuronal genes by the precursor dsRNA. RT-PCR analysis of RNAs from cells with precursor dsRNA and NRSE smRNA was performed with specific primers against genes that were regulated by NRSE-NRSF/REST transcriptional machinery. C) Chromatin IP (ChIP) analysis for the NRSE site on the GluR2 promoter region. PCR primers were designed to hybridize the region to surround the NRSE sequence on rat GluR2 promoter. D) Knock down of precursor RNA for NRSE smRNA by β-catenin RNAi. RT-PCR analysis was performed by using RNA extracts from cells infected with control lentivirus and the lentivirus of β-catenin RNAi. E) Northern blotting analysis showed the down-regulation of NRSE smRNA by β-catenin RNAi.

FIG. 10. Chromatin structure at NRSE sites surrounded by LINEs. ChIP analysis for NeuroD1, LINE1 and NRSE on LINE-NRSE-LINE site on rat chromosome 10 was done with PCR primers that surround the LEF/Sox DNA regulatory element in the NeuroD1 promoter and for the ORF2 region of LINE1. Primers surrounding NRSE sequence on precursor RNA production site on the rat chromosome 10 LINE-NRSE-LINE locus were also prepared.

FIG. 11. Schematic representation of transcriptional control for NeuroD1, LINE1 and NRSE smRNA in adult neurogenesis. The transcriptional regulation occurs on the LEF/Sox DNA regulatory element as a key molecular switch from Sox2-mediating repressor complex in neural stem cells to β-catenin-mediating activator complex in neuroblast cells. The NRSE sequence surrounded by LINEs (LINE-NRSE-LINE) is expressed as long non-coding RNAs in both sense and antisense directions as precursor dsRNA. These precursor long dsRNAs are processed by specific or general dsRNA-recognizing RNases existing in the nucleus, producing NRSE smRNAs, which in turn regulate transcription of neural specific genes.

### SUMMARY OF THE SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. In the accompanying sequence listing:

SEQ ID NO:1 is the polynucleotide sequence of an exemplary transcription control region comprising a plurality of LEF/Sox overlapping response elements.

SEQ ID NO:2 is the polynucleotide sequence of a LEF/Sox overlapping response element.

SEQ ID NOs:3 and 4 are forward and reverse primers (respectively) for amplification of GAPDH.

SEQ ID NOs:5 and 6 are forward and reverse primers (respectively) for amplification of Sox2.

SEQ ID NOs:7 and 8 are forward and reverse primers (respectively) for amplification of NeuroD1.

SEQ ID NOs:9 and 10 are forward and reverse primers (respectively) for amplification of NeuroD2.

SEQ ID NOs:11 and 12 are forward and reverse primers (respectively) for amplification of LEF 1.

SEQ ID NOs:13 and 14 are forward and reverse primers (respectively) for amplification of TCF4.

SEQ ID NOs:15 and 16 are forward and reverse primers (respectively) for amplification of β-catenin.

SEQ ID NO:17 is the NRSE consensus sequence.

SEQ ID NOs:18 and 19 are forward and reverse primers (respectively) for amplification of a LEF/Sox overlapping response element in the NeuroD1 promoter (Example 5).

SEQ ID NOs:20 and 21 are forward and reverse primers (respectively) for amplification of a LEF/Sox overlapping response element in the NeuroD2 promoter (Example 5).

SEQ ID NOs:22 and 23 are the primers for reverse transcription of the sense and antisense strands (respectively) of a LINE-NRSE-LINE element (1).

SEQ ID NOs:24 and 25 are the primers for reverse transcription of the sense and antisense strands (respectively) of a LINE-NRSE-LINE element (2).

SEQ ID NOs:26 and 27 are the primers for reverse transcription of the sense and antisense strands (respectively) of a LINE-NRSE-LINE element (3).

SEQ ID NOs:28 and 29 are forward and reverse primers for Chromatin Immunoprecipitation (Example 16).

SEQ ID NOs:30 and 31 are forward and reverse primers for amplification of a LINE NRSE sequence on rat chromosome 10 (Example 18).

### DETAILED DESCRIPTION

I. Introduction

To initiate neurogenesis, neural stem cells must exit their undifferentiated state and commit to becoming neuroblasts. The present disclosure describes a transcriptional regulatory “master switch” that determines whether the genetic program that mediates neural differentiation is repressed or activated. Surprisingly, the switch from repression to activation of neural specific genes is made via the same DNA regulatory element, designated the “LEF/Sox overlapping element” or “LEF/Sox element,” within the transcription regulatory regions of neural specific genes, such as NeuroD1 and NeuroD2. This transcriptional control element, WWCAAWG (where W can be either A or T) in either orientation, constitutes the substrate for a molecular switch between the Sox2 suppressor complex and TCF/LEF activator complex, regulating repression of neural specific genes in stem cells, and conversely, their activation in neuroblasts. Activation via this molecular switch induces neurogenesis in neural stem cells and controls the irreversible commitment step from stem cells to neuroblasts.

One aspect of the disclosure relates to compositions and methods for expressing polynucleotide sequences in cells. Recombinant nucleic acids are described that include a polynucleotide sequence that is to be expressed, operably linked to a transcription control sequence including one or more LEF/Sox overlapping response elements. For example, the transcription control sequence can include one LEF/Sox overlapping response element or the transcription control sequence can include more than one, such as from about two to about ten, LEF/Sox overlapping response elements. In an embodiment, the transcription control sequence includes five LEF/Sox overlapping response elements. Typically, the transcription control sequence also includes a promoter. In an embodiment, the promoter is a CMV immediate early promoter, e.g., a CMV minimal promoter.

For example, the polynucleotide sequence to be expressed can be a heterologous polynucleotide sequence. That is, the polynucleotide sequence operably linked to the transcription control sequence including the LEF/Sox overlapping response element(s) can be any sequence not ordinarily found in proximity to or under the transcription regulatory control of these response elements in a naturally occurring nucleic acid molecule. The polynucleotide sequence can also be a sequence that, while naturally occurring in proximity to a transcription control sequence with LEF/Sox overlapping response elements, is present in the recombinant nucleic acid in a different conformation, or adjacent to or contiguous with a polynucleotide sequence with which it is not normally associated in a naturally occurring nucleic acid molecule.

In an embodiment, the heterologous polynucleotide sequence encodes a reporter. For example, the heterologous polynucleotide to be expressed can encode a fluorescent polypeptide and/or a polypeptide with enzymatic activity. The fluorescent polypeptide can be a green fluorescent protein (GFP) or a homologue or variant thereof (such as a variant that emits light in the blue, yellow or even red portion of the spectrum). The enzymatic activity of the reporter polypeptide can convert a fluorogenic or chromogenic substrate into an optically detectable product. Exemplary reporters with enzymatic activity include luciferase, β-galactosidase, β-glucuronidase, and chloramphenicol acetyl-transferase.

In alternative embodiments, the heterologous polynucleotide includes an open reading frame (ORF) that encodes a polypeptide or protein, which confers at least one desired property or characteristic when expressed in a cell. Such polypeptides include, for example, structural proteins, enzymes, transcription factors or other nucleic acid binding proteins, antigenic polypeptides, dominant-negative polypeptides, fusion polypeptides (such as the Engrailed and VP16 fusion proteins described herein), reporters, etc.

In other embodiments, the heterologous polynucleotide encodes a functional RNA product. For example, the heterologous polynucleotide can encode a double stranded (ds) modulatory RNA, an inhibitory RNA (iRNA), a small inhibitory RNA (siRNA), an antisense RNA (asRNA) and/or a ribozyme. In an embodiment the recombinant nucleic acid includes a heterologous polynucleotide that encodes a small NRSE dsRNA. In one such embodiment, the heterologous polynucleotide encoding the RNA is flanked on either side by transcription control sequences including one or more LEF/Sox elements (e.g., along with a promoter), such that both the sense and antisense strands of the encoded polynucleotide are transcribed.

In an embodiment, the recombinant nucleic acid is a vector. The nucleic acid can be an autonomously replicating vector, such as a plasmid. In one embodiment, the vector includes the polynucleotide sequence of SEQ ID NO:1.

The recombinant nucleic acids described above are useful in methods for expressing a selected polynucleotide sequence in a cell. Methods for expressing polynucleotides in cells involve introducing a nucleic acid including the selected polynucleotide sequence into a cell. The selected polynucleotide sequence is operably linked to a transcription control sequence that includes one or more LEF/Sox overlapping response elements. The cell into which the selected polynucleotide was introduced (i.e., the host cell) is then grown under conditions in which a protein complex including TCF/LEF binds to the LEF/Sox overlapping response element(s). Upon binding to the response element(s), TCF/LEF induces transcription of the selected polynucleotide sequence in the host cell or one or more progeny cells resulting from division of the host cell.

In an embodiment, the cell has at least one additional heterologous nucleic acid. The additional heterologous nucleic acid favorably encodes a polypeptide that modulates gene expression. The polypeptide can modulate expression by itself, or it can modulate gene expression as a component of a multi-protein complex. For example, the additional heterologous nucleic acid can encode a nucleic acid binding factor, e.g., β-catenin, CREB-binding protein (CBP), a T cell factor (TCF), such as Tcf3 or Tcf4, or a lymphocyte enhancer-binding factor (LEF), such as Lef1. The cell can contain more than one additional heterologous nucleic acid, optionally any other heterologous nucleic acids are selected from among the exemplary factors given above.

In one embodiment, the nucleic acid including the selected polynucleotide sequence is introduced into an undifferentiated cell, such as a stem cell, e.g., an embryonic stem cell (ES) cell or a neural stem cell. Alternatively, the undifferentiated cell can be an oocyte, a zygote. In another embodiment, the nucleic acid is introduced into a neural lineage cell.

In certain embodiments, the cell into which the nucleic acid is introduced is grown under conditions that cause the cell, or at least one progeny thereof, to differentiate into a neural linage cell. In such embodiments, the method directs neural specific expression of the selected polynucleotide. Typically, such neural specific expression includes expression in one or more neural lineage cells. An exemplary neural lineage cell is a neuron, such as a hippocampal neuron. For example, undifferentiated stem cells (e.g., neural stem cells and/or ES cells) can be induced to differentiate into neural cells by growing (culturing) them in the presence of retinoic acid (RA) and/or forskolin (FSK).

Another aspect of the disclosure relates to methods for modulating (e.g., inducing or inhibiting) differentiation of stem cells (such as neural stem cells) into neural lineage cells. In the methods described herein, polypeptides that bind to LEF/Sox overlapping response elements are expressed in cells. Binding of the expressed polypeptide to the LEF/Sox overlapping response element(s) modulates (e.g, induces, inhibits, or prevents) differentiation of the stem cell into a neural lineage cell, depending on the nature of the polypeptide. Typically, polypeptides that bind to LEF/Sox overlapping response elements are expressed in the cell by introducing a polynucleotide sequence encoding the polypeptide operably linked to a suitable transcription control sequence. For example, the transcription control sequence usually includes a promoter, and optionally contains includes additional elements, such as enhancers. For example, the promoter can be a “constitutive” promoter, i.e., a promoter that results in constitutive expression of the encoded polypeptide.

For example, if induction of differentiation is desired, a polypeptide that binds to the LEF/Sox overlapping response elements and activates transcription of nearby polynucleotide sequences is expressed. In one embodiment, the polypeptide that promotes differentiation is a Sox2-VP16 transactivator fusion protein. In another embodiment, the polypeptide that promotes differentiation is β-catenin, for example, phosphorylated (e.g., constitutively phosphorylated) β-catenin. In yet other embodiments, the polypeptide is a TCF or LEF polypeptide.

In contrast, if prevention or inhibition of differentiation is desired (for example, to maintain a stem cell in an undifferentiated state), a polypeptide that binds to the LEF/Sox overlapping response elements and represses transcription of nearby polynucleotide sequences is expressed. In an embodiment, the polypeptide that inhibits differentiation is a Sox2-Eng (engrailed) repressor fusion protein. Cells expressing such an inhibitory polypeptide do not differentiate, even in the presence of agents that would otherwise induce differentiation of the cell, such as RA and/or FSK.

Another feature of the disclosure relates to methods for identifying compositions or agents that regulate (or modulate) differentiation of stem cells into neural lineage cells. Such methods can be cell based, or can be cell free, utilizing cell extracts or purified proteins and nucleic acid substrates.

In an embodiment, agents that modulate or regulate differentiation of stem cells are identified by contacting a nucleic acid comprising a polynucleotide sequence including one or more LEF/Sox overlapping response elements with a reaction mixture. The reaction mixture contains at least one binding factor that binds to the LEF/Sox overlapping response element. Typically, the reaction mixture contains at least one of β-catenin, Lef transcription factors, Tcf transcription factors, CREB-binding protein (CBP), C-terminal-binding protein-1 (ctBP1), and histone deacetylase-1 (HDAC1). The reactions mixture also contains at least one candidate agent, that is, a composition, the effect of which is being determined. The ability of the agent to regulate differentiation is then measured by detecting a change in binding of at least one component of the reaction mixture to the nucleic acid. Typically, a change in binding of the component of the reaction mixture in the presence of the agent is detected in comparison to binding of the component of the reaction mixture in the absence of the agent.

In certain embodiments, the reaction mixture includes recombinant proteins corresponding to multiple binding factors that bind to the LEF/Sox overlapping response element. Such binding factors can include, for example, β-catenin, Lef1, Tcf3, Tcf4 and CBP. In certain embodiments, the reaction mixture includes a soluble extract of a cell.

In another embodiment, a cell containing a polynucleotide sequence including one or more LEF/Sox overlapping response elements is contacted with a candidate agent and the effect of the agent is determined. For example, the ability of the agent to regulate differentiation of a stem cell into a neural cell is determined by preparing a soluble extract of the cell, and detecting binding of a component of the extract to the LEF/Sox overlapping response element(s). The cell can be a stem cell, such as a neural stem cell. Alternatively, the cell can be essentially any cell, as long as, the cell expresses, or the cell extract contains binding factors that bind to the LEF/Sox overlapping response element.

The ability of the agent to regulate differentiation of a stem cell into a neural cell can be determined by detecting binding of a protein complex including β-catenin (e.g., phosphorylated β-catenin) and/or Tcf/LEF to the nucleic acid including the LEF/Sox overlapping response element(s). Such an agent is predicted to induce differentiation of the stem cell into a cell of the neural lineage, e.g., a cell committed to the neural lineage. Alternatively, the ability of the agent to regulate differentiation of a stem cell can be determined by detecting binding of a protein complex including Sox2. An agent that increases binding of Sox2 (for example, by increasing the binding of Sox2 or by decreasing the replacement of Sox2 by other protein factors) is predicted to inhibit or prevent differentiation of stem cells into neural lineage cells. For example, such an agent is predicted to inhibit differentiation of stem cells in response to agents that would otherwise induce differentiation (such as RA and FSK). In some instances, the ability of the agent to regulate differentiation of stem cells can be determined directly by detecting binding of the agent to the nucleic acid with the LEF/Sox overlapping response element(s). The binding can be detected according to any method known to those of ordinary skill in the art for the detection of nucleic acid binding. For example, binding can be detected by detecting a mobility shift during electrophoresis in the nucleic acid including the LEF/Sox overlapping response element(s). Optionally, the nucleic acid includes a polynucleotide sequence that encodes a reporter operably linked to the polynucleotide sequence that includes the LEF/Sox overlapping response element(s).

In one embodiment, agents that regulate differentiation of stem cells into neural lineage cells are identified by contacting a cell with a candidate agent (a composition). The cells contain a polynucleotide sequence that encodes a reporter. This polynucleotide is operably linked to a transcription control sequence containing one or more LEF/Sox overlapping response elements. In an embodiment, the transcription control sequence includes at least two (e.g., a plurality of) LEF/Sox overlapping response elements. For example, the transcription control sequence can include five LEF/Sox overlapping response elements. The ability of the agent to regulate differentiation of stem cells into neural lineage cells is determined by detecting a relative change in expression of the reporter. The relative change in the reporter is typically measured in comparison to a control cell (which also contains the polynucleotide that encodes a reporter) that was not contacted with the agent. For example, agents or compositions can be identified that modulate expression of neural lineage cell specific genes, such as NeuroD1.

In an embodiment, the cells for identifying (e.g., screening) agents are stem cells, such as embryonic stem cells (ES) or neural stem cells. Alternatively, any cells, typically undifferentiated cells, can be utilized, as long as the cell expresses (e.g., endogenously expresses, or expresses a heterologous nucleic acid encoding) LEF/Sox overlapping response element binding factors. Typically, the cell contacted with the agent (the test cell) and the control cell are the same cell type, such as subsets of the same population of cells or cells selected or derived from the same cell line. Thus, for example, the cell contacted with the agent and the control cell can both be stem cells, such as ES cells or neural stem cells. In an embodiment, the neural stem cells are hippocampal neural stem cells that differentiate into neuroblasts.

A relative increase in expression of the reporter can be detected, thereby identifying an agent that induces differentiation of stem cells into neural lineage cells. A relative increase can be an absolute increase in the amount or level of expression of the reporter in the test cell. Alternatively, a relative increase in the reporter can be measured as a constant level of expression of the reporter in the test cell as compared to a decrease in expression of the reporter in the control cell under comparable conditions. Similarly, when an absolute increase in expression of the reporter is detected in the test cell, an absolute increase in the reporter in the control cell can also be detected. As long as the increase in the test cell is greater than the increase in the control cell a relative increase in the reporter is detected.

A relative decrease in expression of the reporter can also be detected. Such a decrease identifies a composition that inhibits differentiation of stem cells into neural lineage cells. A relative decrease in reporter expression in a cell exposed to an agent can be either an absolute decrease in expression or a constant level of expression measured in comparison to an increase in expression in a control cell that is not exposed to the agent. Typically, a relative decrease in reporter expression is detected in a test cell after exposure to a stimulus that would otherwise induce an increase in reporter expression (e.g., concurrently with differentiation of the stem cell into a neural lineage cell) in the absence of the agent. Retinoic acid (RA) and forskolin (FSK) are examples of stimuli that induce differentiation of stem cells into neural lineage cells. Thus, in the presence of RA and/or FSK, an agent that results in a constant level of reporter expression in a test cell contacted with an agent, measured in comparison to an increase in reporter expression in a control cell, is predicted to have an inhibitory effect on differentiation of stem cells into neural lineage cells. Other such stimuli include nucleic acid binding factors, such as β-catenin, Lef transcription factors (e.g., Lef1), Tcf transcription factors (e.g., Tcf3, Tcf4), CBP, glycogen synthase kinase 3 (GSK3) and wnt family activators.

The methods for identifying agents that regulate or modulate differentiation of stem cells into neural lineage cells are particularly suited as screening methods for evaluating libraries of compositions including candidate agents. Typically, a library to be screened will include at least 100 compositions. More commonly, the library includes at least 1000 potential agents, such as, at least 5000 compositions, at least 10,000 compositions, at least 50,000 compositions or even 100,000 compositions or more. One of ordinary skill in the art will appreciate that high-throughput methods can be favorably utilized for screening large composition libraries to identify agents that modulate differentiation of stem cells into neural lineage cells. A composition library can include any variety of compounds, composition, agents and the like. For example, the library to be screened can include natural products, chemical compositions, biochemical compositions, polypeptides, peptides, antibodies, nucleic acids, antisense RNAs, iRNAs, siRNAs, dsRNAs, ribozymes, etc. These categories are not intended to be mutually exclusive, and are provided as non-limiting examples.

Any of a variety of reporters can be utilized in the context of the methods disclosed herein. Examples of suitable reporters include fluorescent polypeptides, such as GFP and its variants (regardless of their emission spectra), as well as polypeptides with enzymatic activity. For ease of detection, reporters with an enzymatic activity that converts a chromogenic or fluorogenic substrate into a visible or fluorescent product are generally utilized. Alternatively, isotopically labeled substrates (that is, substrates labeled with a radioactive isotope) that yield a radiolabeled product can be utilized. Such reporters include β-galactosidase, β-glucuronidase, and chloramphenicol acetyl transferase. Depending on the nature of the reporter, its expression can be monitored, and relative changes in its expression can be optically detected. Optical detection methods include flow cytometry, a variety of automate and semi-automated plate reader formats, microfluidic devices, etc. In an embodiment, the reporter is a selectable marker. Relative increases and decreases in a selectable marker can be detected as increased resistance and sensitivity, respectively, to a selection agent, such as an antibiotic or toxin. Of course, relative changes in reporter expression can be monitored by detecting increases and/or decreases in the RNA from which the reporter is translated. Methods for quantitatively assessing RNA (e.g., mRNA) include, for example, quantitative reverse transcription-polymerase chain reaction (rtPCR) and real time PCR methods, as well as northern analysis and other RNA blotting methods.

II. Abbreviations and Terms

| | |
|---|---|
| 5AzaC | 5′-aza-cytidine |
| asRNA | antisense RNA |
| bHLH | basic helix-loop-helix |
| CAT | chloramphenicol acetyltransferase |
| CBP | CREB-binding protein |
| ChIP | chromatin immunoprecipitation |
| CMV | cytomegalovirus |
| ctBP1 or CtBP1 | C-terminal-binding protein-1 |
| d2EGFP | destabilized enhanced green fluorescent protein |
| ds | double stranded |
| dsRNA | double stranded RNA |
| EC cell | embryonic carcinoma cell |
| EGPF | enhance green fluorescent protein |
| EMSA | electrophoretic mobility shift assay |
| ES Cell | embryonic stem cell |
| FGF2 | fibroblast growth factor 2 |
| FACS | fluorescence-activated cell sorting |
| FRET | fluorescence resonance energy transfer |
| FSK | forskolin |
| GFP | green fluorescent protein |
| GSK3 | glycogen synthase kinase 3 |
| HDAC1 | histone deacetylase-1 |
| IRES | internal ribosome binding site |
| iRNA or RNAi | inhibitory RNA |
| LEF or Lef | lymphocyte enhancer-binding factor |
| LINE | long interspersed nuclear element |
| LTR | long terminal repeat |
| MBDs | methyl-CpG binding domain proteins |
| MeCPs | methyl-CpG binding proteins |
| NRSE | neuron-restrictive silencer element |
| NRSF | neuron-restrictive silencing factor |
| ORF | open reading frame |
| Q-PCR | quantitative PCR |
| RA | retinoic acid |
| RE1 | repressor element 1 |
| REST | repressor element 1 silencing transcription factor |
| rtPCR | reverse transcription polymerase chain reaction |
| SEAP | human placental alkaline phosphatase |
| siRNA | small inhibitory RNA |
| smRNA | small modulatory RNA |
| TCF | T cell factor |
| TSA | trichostatin A |
| TUJ1 | beta-tubulin III protein |
| UTR | untranslated region |

Unless otherwise explained, all technical and scientific terms used herein have the same meaning as commonly understood by one of ordinary skill in the art to which this invention belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: A Comprehensive Desk Reference, published by VCH Publishers, Inc., 1995 (ISBN 1-56081-569-8).

The singular terms “a,” “an,” and “the” include plural referents unless context clearly indicates otherwise. Similarly, the word “or” is intended to include “and” unless the context clearly indicates otherwise. The term “comprising” means “including.” It is further to be understood that all base sizes or amino acid sizes, and all molecular weight or molecular mass values, given for nucleic acids or polypeptides are approximate, and are provided for description. The abbreviation, “e.g.” is derived from the Latin exempli gratia, and is used herein to indicate a non-limiting example. Thus, the abbreviation “e.g.” is synonymous with the term “for example.”

In order to facilitate review of the various embodiments of the invention, the following explanations of specific terms are provided:

A “stem cell” is an undifferentiated cell, capable of indefinite proliferation and generation of differentiated progeny cells. The term “progeny” of a cell or “progeny cell” refers to a cell generated by one or more cycles of DNA replication and division of a parental cell. In mammalian cells, for example, a single cycle of replication and division typically gives rise to two progeny cells. Subsequent cycles of replication and division give rise to exponentially increasing numbers of progeny cells from a single parental or progenitor cell. The progeny of a stem cell can include additional stems cells. Progeny of a stem cell can also include cells of one or more cell lineages or differentiated phenotypes.

Stem cells can be divided into three broad categories on the basis of the variety of differentiated progeny generated by the stem cell. “Totipotent” stem cells, e.g., blastomeres, can give rise to every cell type of an organism. “Pluripotent” stem cells give rise to differentiated cells of any of the three germ layers. “Multipotent” (or “unipotent”) stem cells give rise to a limited set of cell types, typically restricted to a single tissue or lineage. Stem cells can be derived or obtained from either embryonic or adult organisms. Embryonic stem (“ES”) cells are cells obtained from the inner mass cells of a blastocyst. The term “adult” stem cell refers to undifferentiated cells within a specific tissue, which can be found in and obtained or derived from either adult or immature, including embryonic organisms that have undergone sufficient organogenesis that distinct multicellular tissues and organs can be identified. Such adult stem cells are often multipotent cells. Thus, a “neural stem cell” is an undifferentiated cell found in or derived from a tissue of the nervous system of an organism. A common source of neural stem cells is the subventricular zone of the brain, although stem cells can be isolated from a variety of brain regions. Within the hippocampus, stem cells are found in substantial numbers in the subgranular zone of the dentate gyrus.

The term “cell lineage” refers to the ancestry (i.e., the progenitor cell and program of cell divisions) of a cell. A “neural lineage cell” is any cell that arises by division of a neural stem cell and is committed to becoming a neural cell, such as a differentiated neural cell or neuron. Thus, a “neural lineage specific gene” is a gene (a polynucleotide sequence) that is specifically or differentially expressed in a neural lineage cell. Neural lineage specific genes include a myriad of structural and enzymatic components of differentiating and mature neural cells, and include for example, transcription factors (such as NeuroD family genes) and cofactors, neurotransmitters and their synthesizing enzymes, neurotransmitter receptors, receptor-associated factors, ion channels, neurotrophins, synaptic vesicle proteins, cell-cycle related genes, transport machinery proteins, anti- and proapoptotic factors, as well as numerous neuron specific enzymatic and structural proteins, such as growth-associated, cytoskeletal and adhesion molecules involved in axonal guidance.

In the context of this disclosure, a “test cell” is a cell that is contacted with a composition for the purpose of identifying an agent that has a biological effect. The term test cell refers to any such cell, without limitation to any particular cell type, lineage or phenotype. A “host cell” is any cell into which at least one heterologous nucleic acid is introduced. Optionally, the introduced nucleic acid can be expressed, e.g., transcribed, and in some cases, translated. The term also includes any progeny of the host cell (or “parental cell”) that include the nucleic acid introduced into the parental cell. It is understood that not all progeny are identical in phenotype to the parental cell. Differences in phenotype can occur by mutation during replication and/or by differentially expressing one or more genes, e.g., a genetic program activated during differentiation. Nonetheless, such progeny are included when the term “host cell” is used. It should be noted that the terms “test cell” and “host cell” are not mutually exclusive, thus, a host cell into which a heterologous nucleic acid is introduced can also be a “test cell” that is contacted with an agent to determine its biological effect.

In the context of the methods disclosed herein, a “control cell” is a cell, generally of like type to a test cell, which has not been contacted with or exposed to the test agent or composition. In the event that the test cell is subjected to a stimulus, such as a stimulus that promotes differentiation, the control cell is typically also subjected to the same stimulus as the test cell.

An “undifferentiated” cell is a cell that lacks structural and functional specialization. Some undifferentiated cells, such as stem cells, possess the capacity of differentiating into (that is, assuming the structural and functional phenotype of) a variety of mature cell types of multiple cell lineages. Other undifferentiated cells, such as neuroblasts, which are dividing precursor cells committed to a neural fate, are restricted in their capacity to differentiate into cells of a single lineage.

The term “polynucleotide” or “polynucleotide sequence” refers to a polymer of a nucleotides of any length. Generally, the term “nucleic acid” is synonymous with “polynucleotide” or “polynucleotide sequence,” unless clearly indicated to the contrary. For convenience, short polynucleotides, typically of less than about 100 nucleotides in length are often referred to as “oligonucleotides.” Similarly, very short polymers of two, three, four, five, or up to about 10 nucleotides in length, can conveniently be referred to as dinucleotides, trinucleotides, tetranucleotides, pentanucleotides, etc. The nucleotides can be ribonucleotides, deoxyribonucleotides, or modified forms of either nucleotide. The term includes single- and double-stranded forms of DNA (deoxyribonucleic acid) and RNA (ribonucleic acid), as well as DNA-RNA hybrids.

The repeating units in DNA (RNA) polymers are four different nucleotides, each of which comprises one of the four bases, adenine, guanine, cytosine and thymine (uracyl) bound to a deoxyribose (ribose) sugar to which a phosphate group is attached. Triplets of nucleotides (referred to as codons) code for each amino acid in a polypeptide, or for a stop signal. The term codon is also used for the corresponding (and complementary) sequences of three nucleotides in the mRNA into which the DNA sequence is transcribed. Unless otherwise specified, any reference to a DNA molecule is intended to include the reverse complement of that DNA molecule. Double-stranded DNA and RNA (dsDNA and dsRNA) have two strands, which can be defined with respect to the products that they encode: a 5′→3′ strand, referred to as the plus or “sense” strand, and a 3′→5 strand (the reverse compliment), referred to as the minus or “antisense” strand. Because RNA polymerase adds nucleic acids in a 5′→3′ direction, the minus strand of the DNA serves as the template for the RNA during transcription. Thus, the RNA formed will have a sequence complementary to the minus strand and identical to the plus strand (except that U is substituted for T). Except where single strandedness is required by context, DNA molecules, although written to depict only a single strand, encompass both strands of a double-stranded DNA molecule.

A “recombinant” polynucleotide includes a polynucleotide that is not immediately contiguous with one or both of the polynucleotide sequences with which it is immediately contiguous (one on the 5′ end and one on the 3′ end) in the naturally occurring genome of the organism from which it is derived. Thus, a recombinant nucleic acid can include polynucleotide sequences that are “heterologous” with respect to each other. A “heterologous” polynucleotide is a polynucleotide that is not normally (e.g., in the wild-type genomic sequence) found adjacent to a second polynucleotide sequence, or that is not normally found within a particular cell, as the reference indicates. A heterologous nucleic acid or a heterologous polynucleotide can be, but is not necessarily, transcribable and translatable. In some embodiments, a heterologous nucleic acid is a cDNA or a synthetic DNA. In other embodiments, the heterologous polynucleotide sequence is a genomic sequence that encodes an RNA transcript. In other embodiments, a heterologous polynucleotide encodes a reporter. Similarly, a recombinant protein is a protein encoded by a recombinant nucleic acid molecule. A recombinant protein may be obtained by introducing a recombinant nucleic acid molecule into a host cell (such as a eukaryotic cell or cell line, such as a mammalian cell or yeast, or a prokaryotic cell, such as bacteria) and causing the host cell to produce the gene product. Methods of causing a host cell to express a recombinant protein are well known in the art (see, e.g., Sambrook et al., Molecular Cloning: A Laboratory Manual, 2nd edition, New York: Cold Spring Harbor Laboratory Press, 1989).

An “isolated” biological component (such as a polynucleotide, polypeptide, or cell) has been purified away from other biological components in a mixed sample (such as a cell or nuclear extract). For example, an “isolated” polypeptide or polynucleotide is a polypeptide or polynucleotide that has been separated from the other components of a cell in which the polypeptide or polynucleotide was present (such as an expression host cell for a recombinant polypeptide or polynucleotide).

The term “purified” refers to the removal of one or more extraneous components from a sample. For example, where recombinant polypeptides are expressed in host cells, the polypeptides are purified by, for example, the removal of host cell proteins thereby increasing the percent of recombinant polypeptides in the sample. Similarly, where a recombinant polynucleotide is present in host cells, the polynucleotide is purified by, for example, the removal of host cell polynucleotides thereby increasing the percent of recombinant polynucleotide in the sample. Isolated polypeptides or nucleic acid molecules, typically, comprise at least 50%, at least 60%, at least 70%, at least 80%, at least 90%, at least 95% or even over 99% (w/w or w/v) of a sample.

Polypeptides and nucleic acid molecules are isolated by methods commonly known in the art and as described herein. Purity of polypeptides or nucleic acid molecules may be determined by a number of well-known methods, such as polyacrylamide gel electrophoresis for polypeptides, or agarose gel electrophoresis for nucleic acid molecules.

A “transcription control sequence” (alternatively, “transcription regulatory sequence” or “transcription regulatory region” or “regulatory region”) is a polynucleotide sequence comprising one or more cis-acting elements that alone or in combination with other cis-acting elements regulate the transcription of an operably linked polynucleotide sequence. A transcription control sequence can include, without limitation, one or more enhancers, silencers, promoters, transcription terminators, origins of replication, chromosomal integration sequences, 5′ and 3′ untranslated regions, exons and introns. Most commonly, at least a portion of a transcription control sequence is situated 5′ of the polynucleotide sequence (such as, a gene) that it regulates. The regulatory region often is often contiguous (at least in part) with the transcribable sequence it controls, although, in a genome, some cis-acting regulatory elements can be tens of kilobases away from the transcriptional start site of the polynucleotide sequence that they regulate. A “cis-acting regulatory element” or “cis-acting element” is a transcription control element that is located on the same nucleic acid molecule as the polynucleotide sequence that it regulates. For example, an enhancer is a cis-acting element that (when bound by appropriate trans-acting factors) induces or increases transcription of an operably linked polynucleotide. Similarly, a silencer is a cis-acting element that (when bound by appropriate trans-acting factors) represses or decreases transcription of an operably linked polynucleotide. Certain transcription control elements, such as the LEF/Sox overlapping response element, are bi-functional transcription control elements that can mediate induction or repression of gene expression depending on the factors that bind to the control element.

A “trans-acting factor” or “binding factor” or “nucleic acid binding factor” is a protein (or multi-protein complex) that specifically interacts with a cis-acting element. The specific interaction, e.g., specific binding, of a trans-acting factor with a cis-acting element modulates transcription of a polynucleotide operably linked to the cis-acting element. For example, the binding of a trans-acting factor to a cis-acting element can initiate, up-regulate, or down-regulate the transcription of an operably linked polynucleotide sequence. A myriad of known transcription factors are examples of trans-acting factors. More particularly, binding factors that interact with the LEF/Sox overlapping response element include Sox2, β-catenin, CBP, ctBP1, HDAC1, Lef (e.g., Lef1) and Tcf (e.g., Tcf3, Tcf4).

The phrase “LEF/Sox overlapping response element” or “LEF/Sox overlapping element” or “LEF/Sox element” refers to a polynucleotide sequence including in the 5′ to 3′ direction, at a minimum, the sequence: WWCAAWG, or its complement CWTTGWW. “W” designates either A or T according to conventional nucleotide designations. In the compositions disclosed herein, the LEF/Sox overlapping response element is typically located 5′ with respect to the transcribable polynucleotide sequence on the sense (or coding) strand of the nucleic acid. It will therefore be appreciated that the LEF/Sox overlapping response element is orientation independent and functions effectively in both the sense and antisense orientations.

A “promoter” is a polynucleotide sequence sufficient to direct transcription of a nucleic acid. Typically, a promoter is situated adjacent (although not necessarily contiguous) to the start site of transcription. A promoter includes, at a minimum, a polynucleotide sequence to which an RNA polymerase can bind and initiate transcription of an operably linked polynucleotide (“minimal promoter”). A polynucleotide including a promoter can also include elements that restrict promoter-dependent expression to selected cells or tissues, or that render expression inducible by external signals or agents; such elements can be located in the 5′ or 3′ regions of the gene. Both constitutive and inducible promoters have been described (see e.g., Bitter et al., Meth. Enzymol., 153:516-544, 1987). Specific, non-limiting examples of promoters include promoters derived from the genome of mammalian cells (e.g., metallothionein promoter) and from mammalian viruses (e.g., cytomegalovirus (CMV) immediate early gene; Rous Sarcoma virus (RSV) long terminal repeat; the adenovirus late promoter; the vaccinia virus 7.5K promoter), as well as from bacteriophage, plants and plant viruses. Promoters can also be produced by recombinant DNA or synthetic techniques.

A first polynucleotide sequence is “operably linked” to a second polynucleotide sequence when the first polynucleotide is in a functional relationship with the second polynucleotide. For instance, a coding sequence is operably linked to a transcription control sequence if the transcription control sequence affects (e.g., regulates or controls) the transcription or expression of the coding sequence. When recombinantly produced, operably linked polynucleotides are usually contiguous and, where necessary to join two protein-coding regions, are in the same reading frame. However, polynucleotides need not be contiguous to be operably linked.

“Expression” refers to transcription of a polynucleotide, and when used in reference to a polypeptide, to translation. Expression is the process by which the information encoded by polynucleotide sequence is converted into an operational, non-operational or structural component of a cell. The level or amount of expression is influenced by cis-acting elements and trans-acting binding factors, which are often subject to the influence of intra- and/or extra-cellular stimuli and signals. The response of a biological system, such as a cell, to a stimulus can include modulation of the expression of one or more polynucleotide sequences. Such modulation can include increased or decreased expression as compared to pre-stimulus levels. Expression can be regulated or modulated anywhere in the pathway from DNA to RNA to protein (and can include post-translations modifications, e.g., that increase or decrease stability of a protein). For example, the cellular response to a stimulus that promotes differentiation of a stem cell into a committed neural lineage cell, includes induction of expression of neural specific genes, such as NeuroD1. It should be noted that different biological systems can respond differently to an identical stimulus.

A polynucleotide sequence is said to “encode” a polynucleotide or polypeptide if the information contained in the nucleotide sequence can be converted structurally or functionally into another form. For example, a DNA molecule is said to encode an RNA molecule, such as a messenger RNA (mRNA) or a functional RNA (such as an inhibitory RNA (iRNA), small inhibitory RNA (siRNA), double stranded RNA (dsRNA), small modulatory RNA (smRNA), antisense RNA (asRNA) or ribozyme, if the RNA molecule is transcribed from the DNA molecule, and contains at least a portion of the information content inherent in the DNA molecule. A DNA or RNA molecule is said to encode a polypeptide, e.g., a protein, if the protein is translated on the basis of a sequence of trinucleotide codons included within the DNA or RNA molecule. Where the coding molecule is a DNA, the polypeptide is typically translated from an RNA intermediary corresponding in sequence to the DNA molecule.

The term “polypeptide” refers to any chain of amino acids, regardless of length or post-translational modification (for example, glycosylation or phosphorylation), such as a protein or a fragment or subsequence of a protein. The term “peptide” is typically used to refer to a chain of amino acids of from about 3 to about 30 amino acids in length. For example an immunologically relevant peptide may be from about 7 to about 25 amino acids in length, e.g., from about 8 to about 10 amino acids.

A “vector” is a nucleic acid as introduced into a host cell, thereby producing a transformed host cell. Exemplary vectors include plasmids, cosmids, phage, animal and plant viruses, artificial chromosomes, and the like. Vectors also include naked nucleic acids, liposomes, and various nucleic acid conjugates. Certain vectors are capable of autonomous replication in a host cell into which they are introduced (for example, vectors having a bacterial origin of replication replicate in bacteria hosts). Other vectors can be integrated into the genome of a host cell upon introduction into the host cell and are replicated along with the host genome. Some vectors contain expression control sequences (such as, promoters) and are capable of directing the transcription of an expressible nucleic acid sequence that has been introduced into the vector. Such vectors are referred to as “expression vectors.” A vector can also include one or more selectable marker genes and/or genetic elements known in the art.

A “reporter” is a molecule that serves as an indicator of a biological activity. In the context of the present disclosure, a reporter serves as an indicator of transcriptional activity unless otherwise indicated. Typically, a reporter is selected for ease of detection, e.g., by optical means. Common reporters include fluorescent proteins, such as green fluorescent protein (GFP) and numerous variants thereof. Other reporters include proteins with enzymatic activities that convert a fluorogenic or chromogenic substrate into a fluorescent or visible product, or that convert an isotopically labeled substrate into a radioactive product. Examples of such enzymatic reporters include firefly luciferase, chloramphenicol acetyltransferase (CAT), β-glucuronidase and β-galactosidase. A polynucleotide encoding a reporter can be operably linked to a transcription control sequence and introduced into cells. If the transcription control sequence is active in the cell, the reporter will be expressed, and its activity can be detected (qualitatively or quantitatively) using techniques known in the art. Reporters also include selectable markers, the activity of which can be measured as relative resistance or sensitivity to a selection agent, such as an antibiotic.

The term “stimulus” is used generally to refer to a source or signal that causes a reaction in a biological system, such as a cell, tissue, or organism. The signal can be, without limitation, chemical, biochemical, biological, electrical, or a combination thereof. In the context of the methods described herein, a non-limiting example of a stimulus is a signal that induces a change in gene expression or promotes differentiation of a cell. For example, stimuli that induce differentiation of stem cells into neural lineage cells include retinoic acid (RA) and/or forskolin (FSK), as well as various transcription and other intracellular factors.

In the context of the present disclosure, a “library,” for example a composition library, a compound library or a library of agents or potential agents, is a collection of compositions, compounds, agents, etc. A library can be restricted to a single class of compounds or can include a variety of differently classified compounds or compositions. A library can be organized and stored as a single collection or dispersed in multiple locations. A “member of a library” or “library member” is a component of such a collection. Libraries can include, without limitation, inorganic compounds, organic compounds (e.g., produced by combinatorial synthesis), natural products, chemical compositions, biochemical compositions (such as nucleic acids, e.g., DNA, RNA, DNA-RNA hybrids, antisense RNAs, dsRNAs, iRNAs, siRNAs, smRNAs and ribozymes, and peptides, polypeptides, fusion polypeptides, proteins, e.g., antibodies, and the like), metabolites, etc.

The terms “transform,” “transduce” and “transfect” are used essentially synonymously to mean “introduce” a nucleic acid into a cell. While one or the other of these terms may be more appropriate than another depending on the cell type (e.g., eukaryotic or prokaryotic cell) and/or the vector, each of these terms is used to indicate that a subject nucleic acid is introduced into a cell, where it is optionally, replicated and/or expressed. Thus, a transformed cell (transfected cell, transduced cell) is a cell into which a nucleic acid molecule has been introduced by molecular biology techniques. As used herein, the term transformation (or its synonyms) encompasses all techniques by which a nucleic acid molecule might be introduced into such a cell, including, without limitation, transfection with viral vectors, transformation with plasmid vectors, and introduction of naked DNA by electroporation, calcium phosphate precipitation, lipofection, ligand-mediated endocytosis of poly-lysine-DNA complex, and particle gun acceleration.

Except as otherwise noted, the methods and techniques of the present invention are generally performed according to conventional methods well known in the art and as described in various general and more specific references that are cited and discussed throughout the present specification. See, e.g., Sambrook et al., Molecular Cloning: A Laboratory Manual, 2d ed., Cold Spring Harbor Laboratory Press, 1989; Sambrook et al., Molecular Cloning. A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999; each of which is specifically incorporated herein by reference in its entirety.

Although methods and materials similar or equivalent to those described herein can be used in the practice or testing of the present invention, suitable methods and materials are described below. Thus, the materials, methods, and examples described herein are illustrative only and not intended to be limiting.

III. Transcriptional Regulation of Neurogenesis

During initiation of neurogenesis, neural stem cells exit the undifferentiated state and commit to becoming neuroblasts. These neuroblast cells express neurogenic bHLH genes and up-regulate the expression of genes conferring definitive neuronal characteristics, leading toward a mature neuronal state. NeuroD1, a proneural bHLH transcription factor, is expressed in adult hippocampal neuroblast cells. This cell population is distinct from either neural stem cells (which are Sox2 positive) or mature neuronal cells (which are NeuN positive). Over-expression of Sox2 significantly reduces NeuroD expression, indicating that Sox2 acts as a transcriptional repressor (as opposed to an activator) of neuronal differentiation genes. Consistently, loss of Sox2 expression in neural stem cells results in a failure of such cells to maintain an undifferentiated state. De-repression of differentiation-inducing genes (such as NeuroD1) in neural stem cells induces exit from the cell cycle and promotes neuronal differentiation. Indeed, over-expression of NeuroD is sufficient to mediate neuronal differentiation in hippocampal neural stem cells.

Adult hippocampal neural stem cells maintain their undifferentiated state when cultured in vitro in the presence of FGF2. These neural stem cells express Sox2 at high levels. Sox2 protein associates with CTBP1 and HDAC1 to produce a repressor complex that interacts with the LEF/Sox overlapping response elements on NeuroD1 (and NeuroD2) promoters in neuronal stem cells.

When FGF2 is withdrawn and replaced by RA+FSK, both NeuroD1 and NeuroD2 genes are promptly up-regulated and cells begin to differentiation toward the neuronal lineage. Forced expression of NeuroD alone is sufficient to promote neuronal differentiation and to suppress glial lineage differentiation (Hsieh et al., J. Cell Biol., 164:111-122, 2004). Thus, Sox2 inhibits neurogenesis (and maintains an undifferentiated state) by repressing differentiation-inducing NeuroD genes in neural stem cells by forming the Sox2/CtBP1/HADC1 repressor complex.

Induction of neuronal differentiation results in dramatic decreases in expression of Sox2 and HDAC1 genes, and redistribution of CtBP1 from nucleus to the cytoplasm. The appearance of β-catenin in the nucleus, and its formation of a multi-protein complex with TCF/LEF and CBP activator protein, along with the concurrent disappearance of the Sox2/CtBP1/HADC1 complex from the LEF/Sox overlapping response element(s) triggers activation of NeuroD1 (and NeuroD2) transcription. The switch of transcriptional regulation between Sox2 repressor complex and TCF/LEF activator complex on the same DNA element in the NeuroD1 (and NeuroD2) regulatory control region is an important determinant of repression and activation of NeuroD genes between stem cells and neuroblasts. The LEF/Sox overlapping response element results in more effective repression by Sox2 in neural stem cells, as well as more substantial activation by TCF/LEF and β-catenin during neurogenesis, than is produced by these same factors binding to the Sox2 or TCF/LEF recognition elements alone.

This single transcriptional element, A/TA/TCAAA/TG in the NeuroD promoter, constitutes the substrate for a molecular switch between the Sox2 suppressor complex and TCF/LEF activator complex, resulting in the induction of neurogenesis in adult neural stem cells.

Neurogenesis requires the expression of numerous genes that contribute to and confer the mature neuronal phenotype. Along with the NeuroD family of bHLH transcription regulators, nuclear localized small modulatory dsRNA (smRNA) coding NRSE sequences activate expression of genes important for neuronal properties.

NRSE small modulatory RNAs (smRNAs) are non-coding double-stranded (ds) RNAs about 20 bp in length that reside in the nucleus and are believed to play a role in mediating neuronal differentiation. NRSE small modulatory RNAs are described in detail by Kuwabara et al., Cell, 116:779-793, 2004, which is incorporated herein by reference for all purposes.

NRSE smRNAs coordinate the regulation of large clusters of genes involved in neurogenesis. These NRSE smRNAs mediate a variety of complex cellular processes, for example, cell fate determination, differentiation, lineage determination, and organ development. NRSE smRNA modulate expression of, for example, ion channels, neurotransmitter receptors and their synthesizing enzymes, receptor-associated factors, neurotrophins, synaptic vesicle proteins, growth-associated-, cytoskeletal-, and adhesion-molecule factors involved in axonal guidance, transport machinery, transcription factors, and cofactors. The benefits of and methods for making and using NRSE smRNAs are described in U.S. patent application Ser. No. 10/857,784, filed May 28, 2004, which is incorporated herein by reference for all purposes.

Modulation of gene expression with a NRSE smRNA capitalizes on the discovery that the presence of a dsRNA can affect the nature of the interaction of a nucleic acid regulatory element with a trans-acting protein. A NRSE smRNA can act as either an inhibitor or an activator of gene expression based on the particular regulatory element and co-factor modulatory proteins with which it interacts. The association between the smRNA and the components of regulatory machinery can be a direct physical association or an indirect association, for example, through an intermediate molecule. For example, the NRSE smRNA, which includes the NRSE/RE1 sequence, interacts with the NRSE/REST transcription factor, a principal bipotential regulator of neuron-specific genes.

In the absence of the NRSE smRNA, neural specific genes are repressed in neuronal stem cells as a result of NRSF binding to the NRSE, and the neuronal stem cells remain at the progenitor stage. Neural specific genes are silenced in the absence NRSE smRNAs by the recruitment of histone deacetylases (HDACs) and particular co-factor modulatory proteins, including, methyl-CpG binding proteins (MeCPs) and methyl-CpG binding domain proteins (MBDs) by the NRSE-bound NRSF. Such co-factor modulating proteins mediate transcriptional repression by associating with additional modulating proteins, for example, members of the Sin3 and histone deactylase protein families. Depending on the particular co-factors recruited, transcription of one or more genes specific to expression in the nervous system is repressed. In the presence of NRSE smRNAs, transcriptional repression is relieved, resulting in activation of neural specific genes.

The NRSFIREST is conserved between Xenopus laevis, Danio rerio, Fugu rubripes, mouse, rat, chicken, sheep, and human. The expression of NRSF/REST has been detected in non-neuronal cells undergoing tissue organization during development, where it restricts neuronal gene expression to the nervous system by silencing neural specific genes in non-neuronal cells. NRSE/REST is also expressed in adult mammalian CNS neurons (Palm et al., J. Neurosci., 18:1280-1296, 1998; Calderone et al., J. Neurosci. 23:2112-2121, 2003; Kuwabara et al., Cell 116:779-793, 2003). The mRNA expression level is elevated in response to ischemic or epileptic insults (Palm et al., J. Neurosci., 18:1280-1296, 1998; Calderone et al., J. Neurosci. 23:2112-2121, 2003), and the interaction with huntingtin was detected in neuronal cells in a mouse model of Huntington's disease (Zuccato et al., Nat. Genet., 35(1):76-83, 2003).

Neuronal genes whose expression is controlled by the NRSE/REST protein carry the NRSE/RE-1, as a cis-acting regulatory element within their DNA. NRSE smRNAs, which appear transiently during early neuronal differentiation, play an important role in the transition from repressor to activator by NRSE/REST. The NRSE sequences are embedded widely in the genomic region, typically in promoters of neuron-specific genes, including ion channels, neurotransmitter receptors and their synthesizing enzymes, receptor-associated factors, neurotrophins, synaptic vesicle proteins, growth-associated and cytoskeletal and adhesion molecule factors involved in axonal guidance, transport machinery, and transcription factors and cofactors. They also serve to direct cells to a neuronal differentiation pathway (Chong et al., Cell, 80:949-957, 1995; Schoenherr and Anderson, Science, 267:1360-1363, 1995; Palm et al., J. Neurosci., 18:1280-1296, 1998; Huang et al., Nat. Neurosci., 2:867-72, 1999). NRSF/REST mediates the transcriptional repression of neuron-specific genes through the association of histone deacetylase (HDAC) complex, MeCP2, or MBD1 in non-neuronal cells (Huang et al., Nat. Neurosci., 2:867-72, 1999; Lunyak et al., Science, 298:1747-1752, 2002; Naruse et al., Proc. Natl. Acad. Sci. USA, 96:13691-13696, 1999; Kuwabara et al., Cell, 116:779-793, 2004), but the appearance of NRSE smRNAs in an early stage of neurogenesis in the adult hippocampus leads to the initiation of transcription of NRSE genes by modulating the function of NRSF/REST from repressor to activator. The NRSE smRNAs appear during a relatively short period in neuroblasts, specifically during the transition from adult hippocampal neural stem cells to their neuronal cell fate. The smRNAs localize only in the nucleus to function as RNA transcriptional modulators during early neurogenesis. When cells differentiate into more mature neurons, the smRNAs gradually disappear from the cells.

Numerous NRSE sequences were found distributed throughout the genome in proximity to retrotransposon LINEs. Close proximity between NRSE sequences and LINEs has been observed in the human, rat and mouse genomes. Although most LINEs have severely truncated forms lacking transposable ability, the 5′ UTR region and partial fragment of LINE, including most of the ORF2 portion, have sufficient function as an inherent promoter to activate nearby genes, even in a truncated form. This inherent promoter element determines the cell-specific expression of NRSE non-coding smRNAs in neuroblasts.

The LINE promoter activity originates in the 5′ untranslated region (UTR) and ORF2 sequences and is mediated via multiple LEF/Sox overlapping response elements in early neuronal differentiation. Through these overlapping DNA regulatory elements, the promoter activity of LINE is controlled, in a manner analogous to that of NeuroD, by the molecular switching from the Sox2/CtBP1/HDAC1 transcriptional repressor complex in neural stem cells to the TCF/LEF1/β-catenin/CBP activator complex in neuroblasts. Many non-coding transcripts containing the genome-embedded NRSE sequences are generated from nearby LINE promoters during early neuronal differentiation resulting in the production of NRSE smRNAs. The resulting smRNAs bind to the NRSF/REST protein contributing to the localization of transcription factors to each NRSE locus. This process induces neurogenesis by activating NRSE-coding neuronal specific genes that contribute to the mature neuronal phenotype.

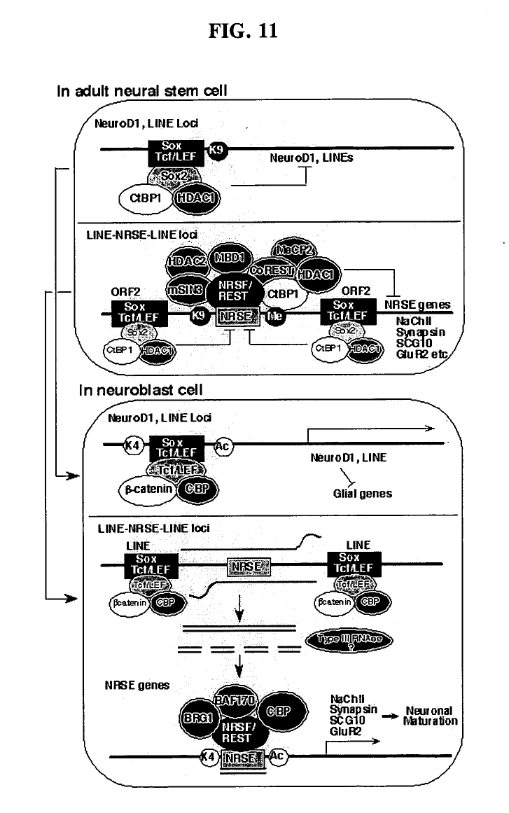

Thus, during early neuronal differentiation, e.g., in the adult hippocampus, transcription of three distinct effectors, NeuroD1, retrotransposon LINEs, and NRSE smRNA, are produced through a coordinated transcriptional regulatory system. This regulatory system is illustrated schematically in FIG. 11. The LEF/Sox overlapping response element serves as the basis for a molecular switch from a Sox2 repressor complex in neural stem cells to a β-catenin activator complex in neuroblast cells. The promoter of NeuroD1 contains the LEF/Sox DNA regulatory elements, allowing neuroblast cells to produce the critical bHLH neurogenic NeuroD1 gene as the dominant neurogenic gene, for example, during adult hippocampal neurogenesis. The intact retrotransposon LINE1 also carries the LEF/Sox overlapping response elements on 5′ UTR and ORF2. Most of the truncated, shorter LINE elements that are scattered throughout the genome also contain the LEF/Sox overlapping response elements in the ORF2 portion, retaining the ability to act as promoters during neurogenesis. These elements mediate production of non-coding as well as coding RNAs in neuroblast cells. The NRSE sequence surrounded by LINEs (LINE-NRSE-LINE) is expressed as long non-coding RNAs in both sense and antisense directions. These precursor long dsRNAs are processed (for example, by specific or general dsRNA-recognizing RNases existing in the nucleus) to produce NRSE smRNAs. Together with NRSF/REST, the generated NRSE smRNAs activate neuron-specific genes to direct cells into functionally mature neurons.

IV. Nucleic Acids Including the LEF/Sox Response Element

One aspect of the present disclosure relates to isolated and/or recombinant nucleic acids that include at least one LEF/Sox overlapping response element. Such nucleic acids are useful, for example, in the context of a transcription control sequence for regulating expression of an operably linked polynucleotide sequence. Numerous LEF/Sox overlapping response elements are found in the genomes of multicellular eukaryotes, particularly mammals, such as rats, mice and humans. As shown herein, the LEF/Sox overlapping response element is an important transcription regulatory element involved in modulating expression of neural specific genes, such as NeuroD1 and NeuroD2, as well as LINE elements and NRSE modulatory RNAs. The LEF/Sox overlapping response element can be used in the context of a transcription regulatory sequence to modulate expression of operably linked polynucleotides, for example, following introduction into cells, tissues or organs.

Thus, in an embodiment, an isolated or recombinant nucleic acid includes a polynucleotide sequence with one or more LEF/Sox overlapping response elements. Such polynucleotide sequences can function as transcription control sequences capable of regulating expression of operably linked nucleic acids. For example, a polynucleotide sequence encoding a polypeptide or RNA with a desirable functional attribute can be ligated directly or indirectly (without or with additional intervening sequences) to a transcription control sequence with one or more LEF/Sox overlapping response elements. In an embodiment the polynucleotide sequence operably linked to the transcription control sequence, and subject to transcription regulation via the LEF/Sox overlapping response element(s) encodes a reporter. Alternatively, polynucleotides that encode polypeptides (or RNAs, e.g., functional RNAs such as siRNAs, smRNAs, antisense RNAs and/or ribozymes) are operably linked to the transcription control sequence with the LEF/Sox overlapping response element(s). For example, polynucleotides that encode therapeutic polypeptides (that is, polypeptides with an effect hat prevents or that can be used to treat a symptom, condition or disease) can be operably linked to a polynucleotide sequence with a LEF/Sox overlapping response element.

Such a transcription control sequence can include a single LEF/Sox overlapping response element, or it can include a plurality of LEF/Sox overlapping response elements. For example, the transcription control sequence can include from two to more than 20 LEF/Sox overlapping response elements. More commonly, such a polynucleotide sequence has from two to ten LEF/Sox overlapping response elements, such two, or three, or four or five LEF/Sox overlapping response elements. The transcription control sequence can also include a promoter, that is, a polynucleotide sequence that serves as a site to which an RNA polymerase binds and initiates transcription. The promoter can include additional transcription regulatory sequences, or it can be a minimal promoter providing the necessary sequences for polymerase binding and initiation of transcription.

Numerous promoters, consistent with transcription in eukaryotic cells, including mammalian neurons and stem cells are known in the art. For example, the cytomegalovirus (CMV) immediate early promoter can favorably be utilized in the context of a transcription control sequence to initiate transcription of an operably linked polynucleotide sequence. Additionally, transcription control sequences containing the promoter and enhancer regions of the SV40 or long terminal repeat (LTR) of the Rous Sarcoma virus are readily available, as are numerous inducible promoters, such as the glucocorticoid-responsive promoter from the mouse mammary tumor virus (Lee et al., Nature, 294:228, 1982). Promoters from cellular genes expressed in neural lineage cells, such as the phosphoglycerate kinase (“PGK”) promoter and the eukaryotic initiation factor (“eIF2”) promoter are also suitable for this purpose.

In one embodiment, the nucleic acid includes (at least) two transcription control sequences, each of which includes one or more LEF/Sox overlapping response elements. Typically, each transcription control sequence also includes a promoter. The two transcription control sequences are arranged on either side (flanking) a transcribable polynucleotide sequence. In such a nucleic acid both the sense and antisense strands can be transcribed from their respective 5′ transcription control sequences.

In an exemplary embodiment, the transcription regulatory sequence includes the sequence of SEQ ID NO:1.

In addition, the transcription control sequence can include specific initiation signals which aid in the efficient translation of the heterologous coding sequence. For example, initiation signals are particularly desirable when the operably linked polynucleotide lacks an endogenous ATG initiation codon, e.g., in a polynucleotide sequence derived from a cDNA, amplification product, or synthetic polynucleotide sequence. Such signals can include, e.g., the ATG initiation codon, as well as adjacent sequences. To insure translation of the operably linked polynucleotide, the initiation signal is inserted in the correct reading frame relative to the encoded product.

In some embodiments, the transcription control sequence including the LEF/Sox overlapping response element(s) is incorporated into a vector comprising additional nucleotide sequences. Suitable vectors include plasmids, viral vectors, cosmids, and artificial chromosomes. In some embodiments, the vector includes a bacterial (and optionally, a eukaryotic) origin of replication that permits autonomous replication when introduced into a bacterial (or eukaryotic) cell. Numerous vectors, for example, expression vectors, are known to those of ordinary skill in the art, and many are available commercially. Alternatively, novel vectors can be assembled de novo from available components, or using synthetic polynucleotide sequences.

If desired, polynucleotide sequences encoding additional expressed elements, such as signal sequences, secretion or localization sequences, and the like can be incorporated into the vector, usually in-frame with the polynucleotide sequence of interest, e.g., to target polypeptide expression to a desired cellular compartment, membrane, or organelle, or into the cell culture media. Such sequences are well known in the art, and include secretion leader peptides, organelle targeting sequences (e.g., nuclear localization sequences, ER retention signals, mitochondrial transit sequences), membrane localization/anchor sequences (e.g., stop transfer sequences, GPI anchor sequences), and the like.

The transcription control sequence can regulate the expression of an operably linked heterologous polynucleotide sequence. Essentially any polynucleotide sequence can be linked in an operative relationship with the transcription control sequences described herein. For example, the polynucleotide sequence can encode a polypeptide with a selected functional attribute. For example, the polypeptide can be a protein with a desired enzymatic activity (e.g., an enzyme involved in synthesis of a neurotransmitter), expression of which is desired in a cell of the neural lineage, such as a neuroblast or mature neuron. Alternatively, the protein can be a structural component of neural cells, such as a transcription factor (for example a NeuroD family transcription factor), a cofactor, a peptide or polypeptide neurotransmitter, a neurotransmitter receptor, a receptor-associated factor, an ion channel, a neurotrophin, a synaptic vesicle protein, a cell-cycle related genes, a protein component of the transport machinery, an anti- or proapoptotic factor, or a protein, such as a growth-associated, cytoskeletal or adhesion molecule protein involved in axonal guidance. In some cases, the polypeptide is selected to modulate the activity of an endogenous polypeptide or protein, for example a polypeptide that is expressed under pathological or disease conditions. Polypeptides that modulate activity of expressed polypeptides include, e.g., dominant negative polypeptides, antibodies and fragments thereof, and other binding proteins that interact specifically with an expressed polypeptide, e.g., including fusion polypeptides incorporating a binding domain with one or more additional functional or structural domains.

Additionally, the heterologous polynucleotide can encode a RNA with a desired functional activity. Exemplary RNA molecules include siRNAs, smRNAs (such as the NRSE smRNAs described herein), antisense RNAs and ribozymes. For example, a functional RNA can be selected to modulate expression and/or activity of a target that is normally expressed in neural lineage cells. In other cases, the functional RNA can interact with a target that is expressed in a pathological or disease state.

For example, a heterologous polynucleotide sequence encoding a NRSE RNA can be operably linked to a transcription control sequence with one or more LEF/Sox overlapping response elements. The polynucleotide sequence can encode either the sense or antisense transcript of a long NRSE RNA precursor RNA. In this context a long NRSE RNA precursor is longer than about 100 nucleotides in length, e.g., from about 100 nucleotides to about 12 kb. Typically, the long NRSE RNA transcript is from about 500 kb to about 10 kb. For example, a polynucleotide encoding a long NRSE RNA precursor (in either the sense or antisense orientation) can be about 1 kb in length. Optionally, the sense and antisense strands can both be transcribed from the same double stranded nucleic acid. For example, a double stranded (sense and antisense) NRSE polynucleotide can be flanked on either side by, and operably linked to, transcription control sequences including one or more LEF/Sox overlapping response elements (and typically, a promoter). A long NRSE RNA precursor is subject to post-transcriptional processing to form NRSE smRNAs. Alternatively, the heterologous polynucleotide encoding an NRSE RNA can encode a synthetic oligonucleotide, such as a synthetic oligonucleotide that folds into a hairpin conformation to produce a double stranded smRNA. Such an oligonucleotide is typically from about 40 to about 100 nucleotides in length, and can include, in addition to the sense and antisense NRSE sequence, a linker ranging in size from about 2 to more than 40 nucleotides in length. Additional details regarding NRSE RNAs can be found, e.g., in the examples section and in U.S. patent application Ser. No. 10/857,784, the disclosure of which is incorporated by reference for all purposes.

The mechanism of action for such functional RNAs is appreciated in the art, and protocols sufficient to guide one of ordinary skill in the design and construction of such functional RNAs are available, e.g., Antisense Technology, Part A, Meth. Enzymol., Vol. 313, ed. by Phillips, Academic Press, 1999; Engelke, RNA Interference (RNAi): The Nuts & Bolts of siRNA Technology, DNA Press, 2004; and Applied Antisense Oligonucleotide Technology, ed. by Stein and King, Wiley-Liss, 1998.

In other embodiments, the heterologous polynucleotide encodes a reporter. Reporters include a variety of molecules that can easily be detected by optical or other means. For example, common reporters include the green-fluorescent protein (GFP) of Aequoria Victoria, Renilla reniformis, and Renilla mullerei and numerous variants thereof with enhanced or altered excitation and/or emission characteristics. Exemplary GFPs suitable as reporters in the context of this disclosure include without limitation GFPs and variants described by Chalfie et al., Science, 263:802-805, 1994; Heim et al., Proc. Natl. Acad. Sci. USA, 91:12501-12504, 1994; Heim et al., Nature, 373:663-664, 1995; Peelle et al., J. Protein Chem., 20:507-519, 2001; and Labas et al., Proc. Natl. Acad. Sci. USA, 99:4256-4261, 2002, and in U.S. Pat. Nos. 6,818,443; 6,800,733; 6,780,975; 6,780,974; 6,723,537; 6,265,548; 6,232,107; 5,976,796; and 5,804,387. Red fluorescent proteins are described in, e.g., U.S. Pat. No. 6,723,537. Such fluorescent proteins can be optically detected using, for example, flow cytometry. Flow cytometry for GFP is described in, e.g., Ropp et al., Cytometry, 21:309-317, 1995, and in U.S. Pat. No. 5,938,738. Other suitable detection methods include a variety of multiwell plate fluorescence detection devices, e.g., the CYTOFLUOR 4000® multiwell plate reader from Applied Biosciences. Other reporters include proteins with enzymatic activities that convert a fluorogenic or chromogenic substrate into a fluorescent or visible product. Examples of such enzymatic reporters include various naturally occurring and modified luciferases. Exemplary luciferases are described in U.S. Pat. Nos. 6,552,179; 6,436,682; 6,132,983; 6,451,549; 5,843,746 (biotinylated); 5,229,285 (thermostable), and 4,968,613. U.S. Pat. No. 5,976,796 describes a luciferase-GFP reporter. Additional examples of reporters with enzymatic activity include, e.g., chloramphenicol acetyltransferase (CAT), β-glucuronidase, β-galactosidase and alkaline phosphatase. Reporters also include selectable markers, the activity of which can be measured as relative resistance or sensitivity to a selection agent, such as an antibiotic. Exemplary selectable markers include thymidine kinase, neomycin resistance, kanamycin resistance, and ampicillin resistance.

A polynucleotide encoding a reporter can be operably linked to a transcription control sequence including one or more LEF/Sox overlapping response elements. When such a reporter construct is introduced into cells (host cells) the reporter is expressed under the regulation of the transcription control sequence, and is expressed under conditions resulting in activation of transcription from the transcription control sequence. Similarly, the reporter is not expressed under conditions resulting in suppression of transcription from the transcription control sequence. Expression of the reporter can be readily detected using procedures known in the art, providing a measure of gene activity regulated by the transcription control sequence. Thus, a reporter construct as described herein is useful for qualitative and/or quantitative monitoring of transcription under the control of LEF/Sox overlapping response elements.

V. Methods of Expressing Polynucleotides

The polynucleotides disclosed herein can be expressed following introduction into host cells. The transfer of DNA into eukaryotic, in particular human or other mammalian cells, is now a conventional technique. The vectors are introduced into the recipient cells as pure DNA (transfection) by, for example, precipitation with calcium phosphate (Graham and vander Eb, Virology, 52:466, 1973) or strontium phosphate (Brash et al., Mol. Cell Biol., 7:2013, 1987), electroporation (Neumann et al., EMBO J., 1:841, 1982), lipofection (Felgner et al., Proc. Natl. Acad. Sci USA, 84:7413, 1987), DEAE dextran (McCuthan et al., J. Natl. Cancer Inst., 41:351, 1968), microinjection (Mueller et al., Cell, 15:579, 1978), protoplast fusion (Schafner, Proc. Natl. Acad. Sci. USA. 77:2163-2167, 1980), or pellet guns (Klein et al., Nature, 327:70, 1987). Alternatively, the cDNA, or fragments thereof, can be introduced by infection with virus vectors. Systems are developed that use, for example, adenoviruses (Ahmad et al., J. Virol., 57:267, 1986), retroviruses (Bernstein et al., Gen. Engr'g., 7:235, 1985), or Herpes virus (Spaete et al., Cell, 30:295, 1982).

Using the above techniques, expressible polynucleotides including one or more LEF/Sox overlapping response elements can be introduced into human cells, mammalian cells from other species or non-mammalian cells as desired. The polynucleotides can be introduced into mammalian neural lineage cells (including, e.g., neuroblasts, differentiating neural lineage cells, and mature neurons) in vitro or in vivo. For example, a polynucleotide including one or more LEF/Sox overlapping response elements can be introduced into primary neural cells in vitro by a variety of procedures including, e.g., electroporation, calcium phosphate precipitation, liposome-mediated transfer, etc. Similarly, polynucleotides including LEF/Sox elements can be introduced into established cells with neural lineage characteristics, such as PC12 cells and F11 cells, and primary or established tumor cell lines of neuroectodermal origin. Such a polynucleotide can be introduced into neural cells in vivo, for example using viral vectors, such as lentivirus vectors, adenovirus vectors or retrovirus vectors.

Following introduction into host cells, the cells are grown under appropriate growth conditions selected for replication, self-renewal, differentiation, etc., as desired. Under appropriate growth conditions, endogenous factors expressed in the neural lineage cells that are involved in regulating transcription via the LEF/Sox overlapping response element(s) can bind to the introduced nucleic acid including a transcription control sequence with one or more LEF/Sox overlapping response elements and repress or activate transcription of an operably linked polynucleotide sequence. For example, the cells can be grown under conditions in which TCF/LEF (along with, e.g., β-catenin and CBP) bind to the overlapping response elements, resulting in expression of the linked polynucleotide sequence.

Expressible polynucleotides with one or more LEF/Sox overlapping response elements can also be introduced into undifferentiated cells. In one instance the undifferentiated cells are stem cells, such as embryonic or adult neural stem cells, e.g., hippocampal neural stem cells, embryonic stem (ES) cells, embryonic carcinoma (EC) cells (such as P19 EC cells). Such cells can be induced to differentiate by growth under conditions, for example including exposure to exogenous agents) that promote commitment and differentiation of neural lineage cells. For example, undifferentiated cells, such as neural stem cells can be grown in the presence of retinoic acid (RA) and/or forskolin (FSK), which promote neural differentiation. In some cases, the polynucleotides can be expressed in terminally differentiated cells of a non-neural lineage, such as fibroblasts.

If expression of a polynucleotide sequence that is operably linked to a transcription control sequence including LEF/Sox elements is desired in a cell that does not normally express one or more factors that bind to the LEF/Sox overlapping response element(s), one or more of such factors can be expressed in the cell by introducing an expression vector encoding the factor. For example, if expression of the polynucleotide is desired in a host cell, such as a stem cell, that does not ordinarily express phosphorylated β-catenin, an expression vector encoding a constitutively active form of β-catenin can be introduced into the cell, e.g., under the transcriptional control of a strong constitutive or inducible promoter. Likewise, expression of other activation factors, such as CBP, TCF (e.g., Tcf3, Tcf4) and LEF (e.g., Lef1) proteins can be produced from heterologous nucleic acids. Similarly, expression of a polynucleotide under the control of one or more LEF/Sox overlapping response elements can be induced by exposing the cells to an agent that increases the expression or activity of one or more of these factors.

In contrast, in the event that repression of a polynucleotide sequence under the control of LEF/Sox elements is desired, such repression can be obtained by expressing (or over-expressing) factors that bind to the LEF/Sox overlapping response elements and result in repression of expression. For example, the Sox transcription factor, HDAC1, and CtBP1 co-repressor protein all contribute to repression of expression via the LEF/Sox overlapping response element.

In some cases, the cells can be introduced (or reintroduced into a subject) for therapeutic purposes. For example, the therapeutic polypeptides (or RNA molecules) operably linked to the polynucleotide sequences with LEF/Sox overlapping response elements can be introduced into neural stem cells (or, e.g., pluripotent stem cells isolated from peripheral blood or bone marrow). The cells can then be introduced into a subject, for example by intrathecal, intraventricular or intraveneous administration, to express the therapeutic agent in situ in the nervous system of the subject. Such methods can be used to express therapeutic polypeptides and/or RNA molecules to treat human and/or veterinary diseases of the nervous system, such as Parkinson's, Alzheimer's, stroke/ischemic injury, ALS, diabetes related neuropathy or retinopathy, etc.

Polynucleotide sequences can also be expressed in neural lineage cells of transgenic mammals, e.g., transgenic mice, by introducing the polynucleotide sequence under the regulatory control of one or more LEF/Sox overlapping response elements into embryos, which then undergo normal (or experimentally disrupted) development and differentiation. Introduction of heterologous nucleic acids into embryos or embryonic stem cells to produce transgenic organisms is well known in the art. For example, a selected polynucleotide sequence under the transcriptional control of one or more LEF/Sox overlapping response elements can be introduced into fertilized oocytes by microinjection, as described in, e.g., Hogan et al., Manipulating the Mouse Embryo, Cold Spring Harbor Press, 1994. The oocytes are implanted into pseudopregnant females, and the litters are assayed for insertion of the transgene. During embryonic (prenatal) and postnatal development, the transgene is subject to expression under the regulatory control of the LEF/Sox overlapping response elements. Thus, the introduced polynucleotide sequence is repressed in non-neural cells and expressed in neural lineage cells. In some cases, the polynucleotide sequence to be expressed encodes a polypeptide with a desired enzymatic or structural attribute, expression of which in neural cells is desired. In other cases, the polynucleotide sequence encodes a polypeptide capable of modulating, e.g., reducing activity or expression of an endogenous gene product. Examples of such polypeptides, which can be expressed under the regulatory control of LEF/Sox elements, include dominant negative polypeptides, fusion polypeptides, antibodies and other binding proteins. Similarly, the polynucleotide can include a functional RNA, the expression of which in neural cells is desirable. Functional RNAs include, for example, antisense RNAs, siRNAs, smRNAs and ribozymes.

Alternatively, expression of RNAs, and optionally polypeptides, expressed under the regulatory control of the LEF/Sox element(s) can also be obtained in a cell-free transcription and/or translation system. The most frequently used cell-free translation systems consist of extracts from rabbit reticulocytes, wheat germ and E. coli. All are prepared as crude extracts containing all the macromolecular components (70S or 80S ribosomes, tRNAs, aminoacyl-tRNA synthetases, initiation, elongation and termination factors, etc.) required for translation of exogenous RNA. Each extract is supplemented with amino acids, energy sources (ATP, GTP), energy regenerating systems (creatine phosphate and creatine phosphokinase for eukaryotic systems, and phosphoenol pyruvate and pyruvate kinase for the E. coli lysate), and other co-factors (Mg²⁺, K⁺, etc.) that facilitate the function of the particular translation machinery. Additionally, for the expression of polynucleotides under the regulatory control of LEF/Sox elements, the extract includes components of the TCF/LEF transcriptional complex, e.g., one or more LEF proteins, TCF proteins, CBP and β-catenin. Commercially available cell-free translation products (also referred to as in vitro translation products) and instructions for use may be purchased from Ambion (e.g., PROTEINscript-PRO™ Kit, RETIC LYSATE IVT™ Kit), Roche Diagnostics (e.g., RTS 500 PROTEOMASTER E. coli HY Kit, RTS 9000 E. coli HY Kit), Qiagen (e.g., EASYXPRESS™ Protein Synthesis Kit), Promega (e.g., TNT® T7 Quick Coupled Transcription/Translation System), and numerous other suppliers.

VI. Methods of Modulating Cellular Differentiation

Another aspect of the disclosure relates to methods of modulating differentiation of stem cells into neural lineage cells. Based on recognition and characterization of the LEF/Sox overlapping response element, methods have been developed to modulate the differentiation of stem cells (such as neural stem cells, e.g., adult hippocampal neural stem cells) into neural lineage cells. In general, the methods involve expressing a polypeptide that binds to a LEF/Sox overlapping response element in a stem cell. Typically the polypeptide is a binding factor, or a component of a multi-protein complex, that specifically binds to the LEF/Sox element and influences transcription of a downstream gene involved in cell fate commitment and/or differentiation, such as the NeuroD genes. For example, expression of binding factors that bind to the LEF/Sox overlapping response elements and activate transcription of such neural differentiation genes, directs stem cells to divide and differentiate into neural lineage cells (that is, induces or promotes neural differentiation). Conversely, expression of factors that bind to the LEF/Sox elements and repress transcription of neural differentiation genes prevents or inhibits neural differentiation.

The polypeptide can be either naturally occurring or engineered. For example, constitutive expression of TCF, LEF or activated β-catenin can be utilized to direct stem cells to differentiate into neural lineage cells. Alternatively, artificial activators, such as a recombinant fusion polypeptide including a Sox2 binding domain fused to the activation domain of a transactivator protein can be utilized to direct or induce stem cells to differentiate into neural lineage cells. One exemplary transactivator fusion protein is VP16 transactivator protein (Sox2-VP16). In contrast, a Sox2 repressor protein including the Sox2 DNA binding domain and a heterologous repressor domain can be used to prevent differentiation of stem cells into neural lineage cells, for example, in the presence of agents such as RA and FSK that would otherwise induce differentiation. One such repressor protein is the Drosophila engrained (Eng) protein.

The relevant polypeptide can be expressed in the cell by introducing a nucleic acid that encodes the polypeptide according to methods well known in the art, e.g., as discussed above. For example, a nucleic acid including a polynucleotide sequence that encodes the polypeptide can be operably linked to strong constitutive, neural specific or inducible promoter and introduced into the cell by electroporation or other previously described method(s). Alternatively, with respect to naturally occurring polypeptides, the cell can be exposed to an agent that induces expression or activation of the endogenous polypeptide.

In other embodiments, functional RNA molecules, which alter the expression of one or more LEF/Sox binding factors, can be expressed in cells to modulate differentiation. For example, expression of β-catenin-specific antisense RNA, siRNA, or ribozymes can be employed to inhibit differentiation of stem cells into neural lineage cells. Similarly, RNAs that diminish expression of Sox2 promote differentiation of stem cells into neural lineage cells.

NRSE smRNAs can also be utilized to promote differentiation of stem cells into neural lineage cells, as described in U.S. patent application Ser. No. 10/857,784. For example, a DNA that encodes a NRSE precursor RNA or an RNA that folds into a hairpin conformation with a ds NRSE component can be expressed under the regulatory control of one or more LEF/Sox overlapping response elements, as described above. Such a nucleic acid can be introduced into cells, such as neural stem cells, to promote neural differentiation.

VII. Screening Methods

Another aspect of the disclosure relates to methods of identifying agents that modulate expression of neural specific genes, such as NeuroD1, and that regulate differentiation of undifferentiated cells into neural cells. Such agents have utility, e.g., in experimental and/or therapeutic applications, for regulating neural gene expression and inducing (or promoting) or preventing (or inhibiting) differentiation of undifferentiated cells, such as stem cells into neural lineage cells. In some instances, agents (such as, compositions or compounds) that modulate differentiation of neural lineage cells can interact directly with LEF/Sox overlapping response elements. Other agents interact with one or more factors that bind to LEF/Sox overlapping response elements, indirectly modulating differentiation. For example, an agent can bind (either transiently or relatively permanently) to the LEF/Sox element or to a factor that binds to the LEF/Sox element, thereby affecting transcription of linked polynucleotide sequences (whether such sequences are endogenous or heterologous). Any agent that has potential (whether or not ultimately realized) to interact directly or indirectly with the LEF/Sox overlapping response element is contemplated for use in the methods of this disclosure.

For example, the methods disclosed herein are useful for screening libraries of agents to identify members that regulate neural gene expression and/or modulate differentiation of cells of the neural lineage. Such agents include, but are not limited to, natural products, chemical and biochemical compositions (such as peptides, polypeptides, and nucleic acids). For example, extracts and/or isolated or purified natural products derived from any of a myriad of sources, e.g., soil, water, microorganisms, plants, animals, can be evaluated for their ability to modulate differentiation of neural lineage cells according to the methods disclosed herein. Similarly, isolated, synthetic peptides such as, for example, soluble peptides, including but not limited to members of random peptide libraries (see, e.g., Lam et al., Nature, 354:82-84, 1991; Houghten et al., Nature, 354:84-86, 1991), and combinatorial chemistry-derived molecular library, for instance, consisting of D and/or L configuration amino acids, or phosphopeptides (including, but not limited to, members of random or partially degenerate, directed phosphopeptide libraries; see, e.g., Songyang et al., Cell, 72:767-778, 1993) can be agents that modulate neural differentiation. In addition, polypeptides, including but not limited to dominant negative polypeptides, fusion polypeptides, and binding proteins (including antibodies, such as, polyclonal, monoclonal, humanized, anti-idiotypic, chimeric or single chain antibodies, and Fab, F(ab′)2 and Fab expression library fragments, and epitope-binding fragments thereof), are all favorably evaluated using the methods disclosed herein. Furthermore, small organic or inorganic molecules (such as members of chemical combinatorial libraries) are also favorably evaluated as potential agents for the modulation of neural differentiation.

Libraries useful for the disclosed screening methods are produced by methods including, but are not limited to, spatially arrayed multipin peptide synthesis (Geysen et al., Proc Natl Acad Sci USA, 81:3998 4002, 1984), “tea bag” peptide synthesis (Houghten, Proc Natl Acad Sci USA, 82:5131 5135, 1985), phage display (Scott and Smith, Science, 249:386-390, 1990), spot or disc synthesis (Dittrich et al., Bioorg. Med. Chem. Lett., 8:2351 2356, 1998), or split and mix solid phase synthesis on beads (Furka et al., Int. J. Pept. Protein Res., 37:487 493, 1997; Lam et al., Chem. Rev., 97:411-448, 1997).

Typically, but not necessarily, high throughput screening methods involve providing a library containing a large number of potential modulators (e.g., specific binding compounds). For example, a library usually includes more than 100 members or compounds. More commonly, a library includes more than about 1000 members. Frequently, a library for use in the methods described herein includes more than about 5000 members, such as more than about 10,000 members. In some cases, the library includes more than about 50,000, more than about 100,000 or even more than about 500,000 or more than about 1 million different members. Such libraries are then screened in one or more assays, as described herein, to identify those library members (such as, chemical species or subclasses) that display a desired characteristic activity (such as, specific binding to, or modulating expression or activity of a neural specific gene, or modulation of differentiation). The compounds thus identified can serve as conventional “lead compounds” or can, themselves, be used as potential or actual therapeutics.

A combinatorial library is a collection of diverse chemical (or biochemical) compounds generated by either chemical synthesis or biological synthesis, by combining a number of chemical “building blocks” such as reagents. For example, a linear combinatorial chemical library, such as a polypeptide library, is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175; Furka, Int. J. Pept. Prot. Res., 37:487-493, 1991; Houghton et al., Nature, 354:84-88, 1991). Other chemistries for generating chemical diversity libraries can also be used. Such chemistries include, but are not limited to: peptides (e.g., PCT Publication No. WO 91/19735), encoded peptides (e.g., PCT Publication No. WO 93/20242), random bio-oligomers (e.g., PCT Publication No. WO 92/00091), vinylogous polypeptides (Hagihara et al., J. Am. Chem. Soc., 114:6568, 1991), nonpeptidal peptidomimetics with glucose scaffolding (Hirschmann et al., J. Am. Chem. Soc., 114:9217-9218, 1992), analogous organic syntheses of small compound libraries (Chen et al., J. Am. Chem. Soc., 116:2661, 1994), oligocarbamates (Cho et al., Science, 261:1303, 1993), and/or peptidyl phosphonates (Campbell et al., J. Org. Chem., 59:658, 1994), nucleic acid libraries (see Sambrook et al., Molecular Cloning, A Laboratory Manual, Cold Springs Harbor Press, N.Y., 1989; and Ausubel et al., Current Protocols in Molecular Biology, Green Publishing Associates and Wiley Interscience, 2001), peptide nucleic acid libraries (see, e.g., U.S. Pat. No. 5,539,083), antibody libraries (see, e.g., Vaughn et al., Nat. Biotechnol., 14:309-314, 1996; and PCT/US96/10287), carbohydrate libraries (see, e.g., Liang et al., Science, 274:1520-1522, 1996; and U.S. Pat. No. 5,593,853), small organic molecule libraries, and the like.

Devices for the preparation of combinatorial libraries are commercially available (see, e.g., 357 MPS, 390 MPS, Advanced Chem Tech, Louisville, Ky.; Symphony, Rainin, Woburn, Mass.; 433A Applied Biosystems, Foster City, Calif.; 9050 Plus, Millipore, Bedford, Mass.). In addition, numerous combinatorial libraries are themselves commercially available (see, e.g., ComGenex, Princeton, N.J.; Tripos, Inc., St. Louis, Mo.; 3D Pharmaceuticals, Exton, Pa.; Martek Biosciences, Columbia, Md.; etc.).

Computer modeling and searching technologies also permit identification of compounds, or the improvement of already identified compounds that can specifically bind to or indirectly modulate gene expression via the LEF/Sox overlapping response element. Examples of molecular modeling systems are the CHARMM and QUANTA programs (Polygen Corporation, Waltham, Mass.). CHARMM performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen et al., Acta Pharmaceutical Fennica, 97:159-166, 1988; Ripka, New Scientist, 54-57, 1988; McKinaly and Rossmann, Ann. Rev. Pharmacol. Toxicol., 29:111-122, 1989; Perry and Davies, OSAR: Quantitative Structure-Activity Relationships in Drug Design, Alan R. Liss, Inc., 1989, pp. 189-193; Lewis and Dean, Proc. R. Soc. Lond., 236:125-140 and 141-162, 1989; and, with respect to a model receptor for nucleic acid components, Askew et al., J. Am. Chem. Soc., 111:1082-1090, 1989. Other computer programs that screen and graphically depict chemicals are available from companies such as BioDesign, Inc. (Pasadena, Calif.), Allelix, Inc. (Mississauga, Ontario, Canada), and Hypercube, Inc. (Cambridge, Ontario). Although these are primarily designed for application to drugs specific to particular proteins, and are thus useful for modeling interactions with factors that bind to the LEF/Sox overlapping response element, they can be adapted to design of drugs that bind specifically to DNA or RNA, such as the agents that bind directly to the LEF/Sox overlapping response element.

Screening methods may include, but are not limited to, methods employing solid phase, liquid phase, cell-based or virtual (in silico) screening assays. The following assays relate to identifying compounds that interact with (e.g., specifically bind to) a factor that binds to the LEF/Sox overlapping response element, compounds that interfere with a protein-protein interaction involving a factor that binds to the LEF/Sox overlapping response element, and to compounds which modulate gene expression via binding to the LEF/Sox overlapping response element. For example, these assays identify compounds that directly bind to, or that bind a factor that binds, a LEF/Sox overlapping response element and modulate expression of neural specific genes (i.e., genes whose expression is differentially expressed in cells of the neural lineage) or modulate differentiation of cells along the neural lineage developmental pathway. Compounds identified via assays such as those described herein may be useful, for example, as agents that regulate expression of neural specific genes and that can be used to modulate differentiation of neural lineage cells. For example, the compounds identified by the methods described herein can be used to treat human and/or veterinary diseases of the nervous system, such as Parkinson's, Alzheimer's, stroke/ischemic injury, ALS, diabetes related neuropathy or retinopathy, etc. Administration of the identified compounds can be accomplished by administering the compounds directly to a subject, for example by intravenous, intraventricular or intrathecal administration routes. Alternatively, stem cells (for example pluripotent stem cells isolated from a subject's peripheral blood or bone marrow) contacted with the compounds can be injected into the ventricles of the brain or administered intrathecally.

Binding Assays

In general, assays used to identify agents that bind to one or more factors that bind to LEF/Sox overlapping response elements, or that bind to the LEF/Sox elements directly, involve contacting a nucleic acid comprising one or more LEF/Sox overlapping response elements in the presence or absence of a LEF/Sox element binding factor with a test agent, under conditions and for a time sufficient to allow the test agent to bind either to the nucleic acid or to a binding factor, thus forming a complex which can be detected. In some instances, an agent that selectively binds to the nucleic acid or to a factor that binds to LEF/Sox overlapping response elements is selected for further testing for its ability to regulate gene expression, modulate cellular differentiation and/or treat at least one symptom of a disorder affecting the brain or other component of the nervous system.

The binding assays can be conducted in a variety of ways. For example, one method to conduct such an assay involves anchoring a nucleic acid including one or more LEF/Sox overlapping response elements, one or more binding factor such as β-catenin, a LEF transcription factor (e.g., Lef1), a Tcf transcription factor (e.g., Tcf3, Tcf4), CBP, ctBP1, HDAC and Sox2, or a test substances onto a solid phase and detecting at least one other assay component bound to the solid phase at the end of the reaction. In one embodiment of such a method, one or more nucleic acids including LEF/Sox elements are anchored onto a solid surface (such as, a microarray or in a microtiter plate), and exposed to a test compound, which is not anchored, in the presence of one or more LEF/Sox binding factors. Optionally, the test compound or the binding factor(s) can be labeled with a detectable moiety. In another embodiment, a plurality of test compounds are attached to the support (for example, in an array or microplate format) and one or more detectable (e.g, labeled) nucleic acid/binding factors are applied to the solid support. In another embodiment, the binding factor(s) is/are anchored to the solid phase support and contacted with the nucleic acid and test agent. Optionally, the test agents and/or binding factors are attached to the solid support at assigned or addressable positions.

In one example method, mixtures of labeled compounds, for instance radiolabeled or fluorescently labeled compounds can be tested for specific binding to isolated binding factors and/or nucleic acids including the LEF/Sox overlapping response element. Substantially purified binding factors and/or nucleic acids are adsorbed onto a solid support (such as, a microarray or in microtiter wells), which may be subsequently blocked with an irrelevant protein, such as casein. Labeled test compounds, such as compounds in one or more of the above described libraries, are separately added such that they are placed in contact with or proximity to the bound factors/nucleic acids. Combinations of labeled compounds can be evaluated in an initial screen to identify pools of candidate agents to be tested individually. This process is easily automated with currently available technology. The reactions are incubated for a time sufficient to permit interaction between the bound molecules and the labeled compounds. The solid support (e.g., microtiter wells or microarray) is washed and the amount of label (such as, radioactivity or fluorescence) measured in the washed wells. Agents that interact with the binding factors or nucleic acids are identified by a change in the amount of label (for instance, radioactivity or fluorescence) present on the solid surface (e.g., in a microtiter well). Typically, the change in binding is detected relative to a control (e.g., in the absence of the agent). For example, the change in binding can be measured as a qualitative difference (e.g., on/off) between the test and control reactions. Alternatively, the change in binding can be measured as a qualitative difference (greater than or less than) between the test and control reactions.

Such agents are optionally isolated and tested in further assays (such as, functional assays) for their ability to regulate gene expression and/or modulate cellular differentiation. Other analogous approaches using beads as a solid support or solution phase screening (e.g., Boger et al., Angew. Chem. Int. Ed. Engl., 42:4138 4176, 1998; Cheng et al., Bioorg. Med. Chem., 4:727 737, 1996) can also be used in this approach.

Briefly, in a solution-phase binding reaction, the target (e.g., a nucleic acid containing LEF/Sox overlapping response elements and optionally one or more binding factor) and a test agent are mixed in solution. Under these circumstances, the target can be purified or present in a mixture of other components, such as in an organ, tissue, or cell extract. Small volumes (such as, in wells of a microtiter plate) can be used to promote high throughput screening. After a period of time to permit binding of an agent to the target, the bound complex is separated from unbound components, and the complexes detected, e.g., by detecting a shift in mobility between bound and unbound targets. One useful way to separate a bound complex is to use a first antibody specific for one or the other of the bound components (such as, an antibody against a LEF/Sox binding factor). The antibody may be bound to a solid support or may be sufficiently large to be separated from other components by centrifugation. A detectable second antibody specific for the bound complex (or the first antibody) is one exemplar method for detecting the separated complex. In this type of assay, the target (such as, the LEF/Sox overlapping element or binding factor) can be purified or present in a mixture of other components, such as in an organ, tissue, or cell extract.

For example, agents that regulate neural specific gene expression and/or modulate differentiation of neural lineage cells can be identified by combining isolated nucleic acids including the LEF/Sox overlapping response elements with one or more purified LEF/Sox binding factors, e.g., β-catenin, a LEF transcription factor (e.g., Lef1), a Tcf transcription factor (e.g., Tcf3, Tcf4), CBP, ctBP1, HDAC and Sox2, in a reaction mixture including, for example, buffers, stabilizing agents, cofactors, energy sources (e.g., ATP, NADH, NADPH), etc, and a test agent. Binding of a component of the reaction mixture to the nucleic acid is then detected as indicated above. Binding of a component of the reaction mixture, such as the test agent or one or more binding factors can be measured as a relative change in the binding as compared to a comparable reaction in the absence of the test agent (that is, a control).

Alternatively, a test agent can be added to a reaction mixture including a soluble extract of a cell that includes a nucleic acid with one or more LEF/Sox overlapping response elements. For example, the extract can be produced from a cell that includes a heterologous nucleic acid that includes the LEF/Sox overlapping response elements. An isolated nucleic acid including the LEF/Sox that can easily be detected, e.g., by attachment of a label or other modification, can be added to the cell extract to facilitate detection of binding. The soluble extract can be prepared from a cell that expresses endogenous LEF/Sox binding factors, such as stem cells, neural lineage cells, immortalized or transformed cell lines with neural characteristics and the like. Typically, the cell is selected to express a desired set of binding factors, such as the proteins that contribute to the Sox2/ctBP1/HDAC1 complex or the proteins that contribute to the β-catenin/Tcf/Lef/CBP complex. In some cases, the cell is transfected with a recombinant construct encoding the desired binding factor(s). Alternatively, one or more additional purified proteins (e.g., proteins expressed from a recombinant nucleic acid) are added to the soluble cell extract. Optionally, the cell is contacted with the test agent prior to preparation of the soluble cell extract.

In some embodiments, binding of a single component, such as a binding factor (for example, β-catenin, a Tcf transcription factor, a Lef transcription factor, CBP, ctBP1, HDAC or Sox2) is detected, e.g., using an antibody specific for the component. Alternatively, a protein complex made up of multiple component proteins, e.g., binding factors can be detected. For example, in an embodiment, a protein complex including β-catenin and Tcf/Lef is detected. In this embodiment, detection of increased binding by a multi-protein complex including β-catenin in the presence of a test agent indicates that the test agent induces (or is likely to induce) transcription of neural specific genes, and/or that the test agent promotes differentiation of stem cells into neural lineage cells. In another embodiment, binding to the nucleic acid of a Sox2 or a multi-protein complex including Sox2 is detected. Binding of a Sox2 complex in the presence of an agent indicates that the agent inhibits neural specific gene expression and/or differentiation along the neural cell lineage. As indicated above, in another embodiment, binding of a test agent directly to the LEF/Sox nucleic acid can be detected. Typically, a test agent that binds directly to a LEF/Sox overlapping response element is further evaluated, e.g., using a method described herein, such as a cellular reporter based assay, to further elucidate its physiological or functional effect.

Thus, agents that directly bind to the LEF/Sox overlapping response element and agents that bind to a LEF/Sox binding factor and alter its capacity to interact with the LEF/Sox overlapping response element can be identified using the methods described herein. Similarly, agents that alter the ability of one or more LEF/Sox binding factor to interact with another component of a multi-protein complex that binds to the LEF/Sox overlapping element can also be identified using these methods. For example, in addition to the methods described above, agents that disrupt protein-protein interaction between components of a LEF/Sox binding complex can be identified, e.g., as described by Boger et al., Bioorg. Med. Chem. Lett., 8:2339 2344, 1998 and Berg et al., Proc. Natl. Acad. Sci. USA, 99:3830 3835, 2002. For example, each of two proteins that are capable of physical interaction (for example, β-catenin and at least one of Tcf, Lef and CBP, or Sox2 and at least one of ctBP1 and HDAC1 or their respective functional fragments) is labeled with fluorescent dye molecule tags with different emission spectra and overlapping adsorption spectra. When these protein components are separate, the emission spectrum for each component is distinct and can be measured. When the protein components interact, fluorescence resonance energy transfer (FRET) occurs resulting in the transfer of energy from a donor dye molecule to an acceptor dye molecule without emission of a photon. The acceptor dye molecule alone emits photons (light) of a characteristic wavelength. Therefore, FRET allows one to determine whether two molecules are interacting or not based on the emission spectra of the sample. Using this system, two labeled protein components are added under conditions where their interaction resulting in FRET emission spectra. Then, one or more test compounds, such as compounds in one or more of the above described libraries, are added to the environment of the two labeled protein component mixture and emission spectra are measured. A decrease the FRET emission, with a concurrent increase in the emission spectra of the separated components indicates that an agent (or pool of candidate agents) has interfered with the interaction between the protein components.

Cellular Assays

Agents that regulate neural specific gene expression and/or that modulate differentiation of neural lineage cells (for example the commitment and differentiation of stem cells into neural lineage cells) can also be identified using cellular assays. For example, such agents can be identified by evaluating expression of a reporter under the regulatory control of one or more LEF/Sox regulatory elements.

In an embodiment, a cell that incorporates a nucleic acid with a polynucleotide sequence that encodes a reporter is contacted with a test agent. The polynucleotide encoding the reporter is operably linked to a transcription control sequence with one or more LEF/Sox overlapping response elements, placing expression of the reporter is under the transcriptional regulatory control the LEF/Sox overlapping response element(s). Nucleic acids in which a polynucleotide encoding a reporter under the regulatory control of a polynucleotide sequence including one or more LEF/Sox overlapping elements are described in detail above and in the Examples below. Following contacting the cell with a test agent, a relative change in expression in the reporter is detected, as compared to a control cell that is not contacted with the test agent. Typically the control cell is a cell of the same type as the cell exposed to the test agent (i.e., the test cell), incorporates the reporter construct, and is grown under the same condition as the test cell.

For example, the test cell and the control cell can be stem cells, such as embryonic stem (ES) cells, or embryonic or adult neural stem cells, such as primary adult hippocampal neural stem cells. For example, the stem cells can be transfected (stably or transiently using known methods as described above) with a construct including the reporter under the regulatory control of one or more LEF/Sox overlapping response elements. Alternatively, the stem cells can be derived from, e.g., an animal or embryo that includes a transgenic reporter construct in which expression of the reporter is subject to transcriptional regulation by the LEF/Sox element(s). In other embodiments, the cells are established (e.g., immortalized or transformed) cell lines, such as embryonic carcinoma (EC) cells, e.g., P19 cells, or PC12 cells, or other cells capable of assuming neural characteristics under at least some conditions. In yet other embodiments, the cells can be cells, including terminally differentiated cells of another lineage, such as fibroblasts. When using cells of a different lineage it is generally desirable to express one or more LEF/Sox binding factor(s) in the cells, for example by stably transfecting a population of cells from which the test and control cells are selected with recombinant construct(s) from which the binding factors can be expressed.

The relative change in expression of the reporter can be a relative increase in expression. A relative increase in expression can be an increase in expression relative to a smaller increase, a constant level or a decrease in expression of the reporter in the control cell. Minor fluctuations in expression in control cells are typically accounted for by experimental error and/or nonspecific effects of cell culture. For example, the relative increase can be an increase in expression of at least 1.5× (times) the level of the reporter in the control cell. Typically the relative increase is at least about 2×, and often at least about 5× or more (for example at least about 10×) greater than a control cell. A relative increase in reporter expression is an indication that the agent induces or promotes expression of neural lineage specific genes and/or promotes commitment and/or differentiation of stem cells into neural lineage cells. Alternatively, the relative change in expression of the reporter can be a relative decrease in expression. For example, a relative decrease in expression of the test cell as compared to a smaller decrease in expression in the control cell, or a constant level in the test cell compared to an increase in the control cell. Such a decrease in expression indicates that the test agent inhibits neural specific gene expression and/or prevents or inhibits differentiation of stem cells into neural cells. For example, a decrease in expression is usually a decrease of at least 1.5× (that is, 1.5× less than) the expression of a control cell. Typically the decrease is at least about 2×, or at least about 5× the level of a control. In some cases the decrease is at least about 10× or more, compared to the control cell.

For example, in an embodiment, the test cell and the control cell are grown in the presence of a stimulus that is known to promote differentiation of stem cells into committed neural lineage cells. Exemplary stimuli include retinoic acid (RA) and forskolin (FSK). When grown in culture with RA and/or FSK, stem cells (e.g., neural stem cells) become committed to development along the neural lineage pathway and begin to differentiate and express a wide variety of neural specific genes, including NeuroD genes, such as NeuroD1 and NeuroD2. Similarly, expression of a reporter under the regulatory control of one or more LEF/Sox elements increases when the cell is grown under conditions (e.g., in the presence of RA and/or FSK) that promote neural differentiation. When such cells are exposed to an agent that inhibits differentiation and/or expression of neural specific genes, expression of the reporter is expected to be decreased as compared to a cell that is not exposed to the agent. Thus, as indicated above, a relative decrease in reporter expression as compared to a control cell indicates that the agent prevents or inhibits differentiation and/or results in repression of neural specific genes. Additional stimuli that promote differentiation of stem cells into neural lineage cells include, for example, β-catenin, Lef transcription factors (such as Lef1), Tcf transcription factor (such as Tcf3 and Tcf4), CBP, glycogen synthase kinase (GSK3), and wnt activators.

Typically such cellular reporter assays involve detecting expression of the reporter at the level of the translated product either by quantitatively measuring reporter protein or by quantifying activity of the reporter. However, it is also possible to evaluate expression of the reporter at the level of transcription directly. Numerous methods for quantitatively evaluating RNA expression are known in the art, and include, for example, northern blotting, dot and/or slot blotting, quantitative PCR methods, transcription assays, and the like. Any of these methods can be employed in the context of the methods disclosed herein to evaluate expression of a reporter. Optionally, such RNA analyses can be performed in high throughput formats, including multiwell plates, microarrays and/or microfluidic formats.

If desired reporter protein can be detected using, for example western blotting or antibody arrays that are suitable for quantitatively measuring protein levels. More commonly, reporter expression is evaluated by measuring an activity of the reporter, such as fluorescence emission by the reporter or enzymatic activity, e.g., enzymatic conversion of a chromogenic or fluorogenic substrate into a colored or fluorescent product. As discussed above, reporters are typically readily detectable or assayable proteins. Numerous reporter genes, including those described above are commonly known, and methods of their use are standard in the art.

In the applicable methods, expression of the reporter is detected using standard techniques for that particular reporter. Most commonly, the reporter is optically detected by detecting the reporter itself (e.g., GFP) or by detecting an optically detectable product formed as a result of an enzymatic activity of the reporter. Exemplary protocols are provided, for example, in the manufacturer's directions for human placental alkaline phosphatase (SEAP), luciferase, or enhance green fluorescent protein (EGPF) available from BD Biosciences (Clontech); or galactosidase/luciferase, luciferase, or galactosidase available from Applied Biosystems (Foster City, Calif., USA); and available from various other commercial manufacturers of reporter gene products, or otherwise known in the art. For example, expression of GFPs can be detected by flow cytometry as described in, e.g., U.S. Pat. No. 5,938,738; Ropp et al., Cytometry, 21:309-317, 1995). Other suitable detection methods include a variety of multiwell plate fluorescence detection devices, e.g., the CYTOFLUOR 4000® multiwell plate reader from Applied Biosciences, and the detection of fluorescent cells using a microfluidic device (such as the 2100 Bioanalyzer from Agilent, Palo Alto) according to Manufacturer's instructions and protocols.

### EXAMPLES

### Example 1

### Culture of Neural Stem Cells

Adult hippocampal neural stem cells were cultured as previously described (Gage et al., Proc. Natl. Acad. Sci. USA, 92:11879-11883, 1995). For neuronal differentiation, cells were cultured in N2 medium (Invitrogen) containing RA (1 μM, Sigma) and Forskolin (5 μM, Sigma). For astrocyte differentiation, cells were cultured with 50 ng/ml BMP-2 (R&D systems), 50 ng/ml LIF (Chemicon) and 1% FCS (Hyclone) for 4 to 10 days. For oligodendrocyte differentiation, cells were cultured in N2 medium after FGF2 withdrawal for 2 to 4 days. Cell imaging was performed using a microscope (Nikon TE300) with a SPOT camera.

### Example 2

### Neural-Specific Expression of Nucleic Acid Comprising LEF/Sox Overlapping Response Elements

A recombinant nucleic acid including a polynucleotide sequence that encodes an optically detectable reporter was ligated downstream (3′) of the transcription regulatory region of the Sox2 gene in the pd2EGFP plasmid (BD Biosciences, Clontech, Palo Alto). Approximately 6 kb of the transcription regulatory region 5′ to the Sox2 coding sequence was ligated 5′ to a polynucleotide sequence encoding d2EGFP (destabilized enhanced green fluorescent protein). The coding sequence additionally contained an internal ribosome binding site (IRES) situated between the sequence encoding the d2EGFP and a polynucleotide encoding puromycin resistance.

Expression of the d2EGFP protein under the transcriptional control of the Sox2 transcription regulatory region was confirmed by introducing the Sox2-GFP construct into multipotent neural stem cells from adult rat hippocampus (Gage et al., Proc. Natl. Acad. Sci. USA, 92:11879-11883, 1995). The transformed cell cultures were expanded with fibroblast growth factor 2 (FGF2). The transformed cells were multipotent stem cells capable of self-renewal in culture with FGF2 and differentiation into various classes of cells in response to various stimuli. For example, the transformed cells differentiated into neurons, oligodendrocytes, and astrocytes after stimulation with RA+FSK (neurons), IGF—I (oligodendrocytes) and 1% FCS (astrocytes), respectively. Adult hippocampal neural progenitor cells also express Sox2 in proliferating culture in the presence of fibroblast growth factor. Expression of a Sox2 promoter-driven d2EGFP was detected in stem cell cultures with FGF2, but not in neural lineage cells (RA+FSK), astrocytes (1% FCS), or oligodendrocyte lineage cells (IGF-1). A reduction in Sox2-GFP expression correlated with differentiation as previously described Palmer et al., Mol. Cell Neurosci., 8:389-404, 1997; Hsieh et al., J. Cell Biol., 164:111-122, 2004; Kuwabara et al., Cell, 116:779-793, 2004). The Sox2 gene is expressed by most progenitor cells and is generally down-regulated by neuronal cells as they exit the cell cycle and differentiate (Bylund et al., Nat. Neurosci., 6:1162-1168, 2003; Graham et al., Neuron, 39:749-765, 2003).

The resulting cell population in included both Sox2-GFP-positive and Sox2-GFP-negative cells. Sox2-GFP-positive cells were cloned by fluorescence-activated cell sorting (FACS). The d2EGFP signal correlated with endogenous Sox2 gene expression.

### Example 3

### Regulation of Transgenic EGFP by the Sox2 Promoter during Neurogenesis in Transgenic Mice

To investigate expression of Sox2 during neurogenesis in neurogenic areas in the adult hippocampus, the Sox2 expression pattern was compared to that of bHLH transcription factors by parallel immunohistochemical staining analysis in the Sox2 promoter-driven EGFP transgenic mouse (D'Amour and Gage, Proc. Natl. Acad. Sci. USA, 100:11866-11872, 2003). NeuroD1-expressing cells were clearly detected in the hippocampal dentate gyrus area, whereas cells expressing Neurogenins 1-3 were not readily detectable. Importantly, expression of Sox2 and NeuroD1 was observed in mutually exclusive subsets of cells. Furthermore, cells expressing either Sox2 or NeuroD1 were negative for S100β (an astrocyte marker) immunostaining. Sox2 positive cells were negative for the mature neuron marker NeuN. Notably, cells expressing NeuroD1 were only detected in the subgranular zone of the dentate gyrus, a location of ongoing neurogenesis in the adult hippocampus. NeuroD1-positive cells co-stained with both α-catenin and TCF, which appeared to be marking the same population of neuroblast cells. These results, combined with earlier reports that NeuroD1 deficiency leads to a complete lack of dentate gyrus formation in mice (Miyata et al., Genes Dev., 13:1647-1652, 1999; Liu et al., Proc. Natl. Acad. Sci. USA, 97:865-870, 2000) indicated that NeuroD1 proteins play a major role in adult hippocampal neurogenesis.

### Example 4

### Transcriptional Regulation of the NeuroD1 Gene during Neurogenesis

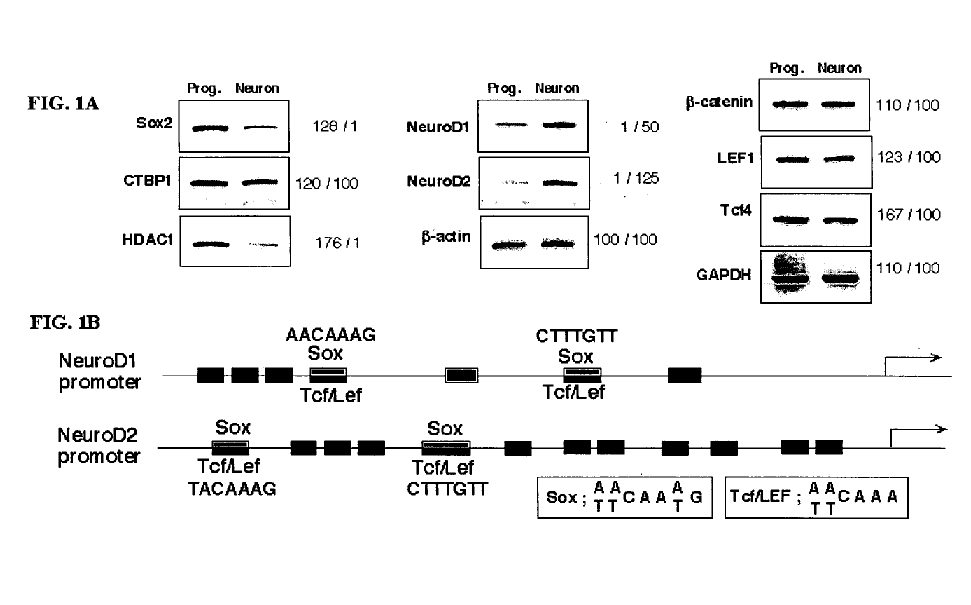

Transcriptional regulation of NeuroD1 during neurogenesis was evaluated in adult stem cells derived from rat hippocampus (Gage et al., Proc. Natl. Acad. Sci. USA, 92:11879-11883, 1995; Hsieh et al., J. Cell Biol., 164:111-122, 2004; Kuwabara et al., Cell, 116:779-793, 2004). Gene expression was evaluated using quantitative RT-PCR, focusing on repressor and activator transcriptional machinery (trans-acting DNA binding factor) genes. After 24 hours of neuronal induction by RA+FSK in adult neural stem cells, NeuroD1 and NeuroD2 gene expression was greatly up-regulated (FIG. 1A). Sox2 and HDAC1 genes were highly expressed in stem cells, and both genes were dramatically down-regulated during neurogenesis. The C-terminal binding protein (CtBP1), a transcriptional co-repressor that has been shown to associate with HDAC1 (Koipally and Georgopoulos, J. Biol. Chem., 275:19594-19602, 2000; Sun et al., Cell, 104:365-376, 2001; Zhang et al., Science, 295:1895-1897, 2002), was expressed in stem cells and during neurogenesis. This CTBP1 co-repressor protein was also reported to bind with TCF/LEF transcription factors (Criqui-Filipe et al., EMBO J., 18:3392-3403, 1999; Hovanes et al., Nucleic Acids Res., 28:1994-2003, 2000; Brunori et al., J. Virol., 75:2857-2565, 2001; Valenta et al., Nucleic Acids Res., 31:2369-2380, 2003; Schmidt et al., Dev. Dyn., 229:703-707, 2004). Gene activation by Wnt/β-catenin signaling requires stabilization of the β-catenin protein and nuclear β-catenin binding with TCF/LEF transcription factors (Moon et al., Science, 296:1644-1646, 2002 (and references therein)). Expression levels of these transcription factors was evaluated and levels of RNA expression of β-catenin, TCF4, and LEF1 were unchanged between stem cells and cells undergoing neurogenesis (FIG. 1A). The numbers on the right side of each panel in FIG. 1A show the relative ratio of expression levels in stem cells and following induction of neurogenesis using quantitative PCR (Q-PCR) with primers corresponding to SEQ ID NOs: 11-16.

Adult neural stem cells were stained using several antibodies known to be expressed during neuronal differentiation in order to evaluate the level of expressed protein and the intracellular localization of these binding factors. The expression of CtBP1 protein was unchanged during neurogenesis but, interestingly, this co-repressor protein changed its localization from the nucleus in stem cells to the cytoplasm during neurogenesis. Concurrent with the localization change of CTBP1, the highest expression level of NeuroD1 protein was detected 24 hrs after neuronal induction, and the expression level gradually decreased as differentiation progressed. In parallel with the transient up-regulation of NeuroD1 at this early stage in neurogenesis, the accumulation of β-catenin protein was observed in both the nucleus and cytoplasm. Although the RNA expression level of β-catenin gene was constitutive during neuronal differentiation, the stability of β-catenin protein was significantly increased during neurogenesis, resulting in nuclear accumulation of the protein. Since the trans-activation of gene expression through TCF/LEF transcription factors is mediated by the nuclear localization of β-catenin protein after accumulation of stabilized β-catenin protein, the correlation between the timing of the highest expression of NeuroD1 and the nuclear localization of α-catenin indicated that the initiation of active transcription of NeuroD1 is triggered through the β-catenin signaling.

### Example 5

### Construction of a NeuroD1-Luciferase Construct and Characterization of the Transcription Regulatory Region of NeuroD1

The DNA binding sequences of both Sox2 and TCF/LEF transcriptional factors on the NeuroD1 and NeuroD2 promoters was analyzed to clarify the transcriptional regulation of NeuroD during the transition from neural stem cells to neuroblast cells. As shown in FIG. 1B, the binding sites of many TCF/LEF transcriptional factors and of several Sox transcription factors were found within 3 kb upstream of the promoter region. Surprisingly, some sequences overlapped with each other, and such overlapping DNA regulatory elements were found in both the NeuroD1 and NeuroD2 promoters. In light of the coordinated expression of NeuroD and β-catenin, and the distinction between NeuroD/β-catenin expressing cells and cells expressing Sox2, it was predicted that these two distinct transcriptional factors, Sox2 and TCF/LEF/β-catenin, regulate NeuroD gene expression, both negatively and positively, through these DNA regulatory elements.

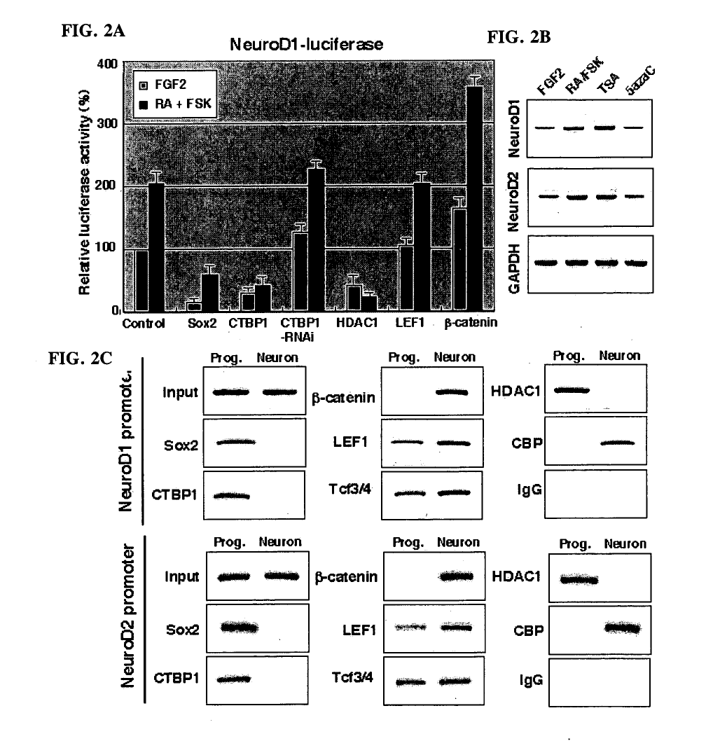

To evaluate the effect of these transcriptional factors on the NeuroD1 promoter, a NeuroD1 promoter-driven luciferase construct was prepared and co-transfected with a Renilla luciferase construct into adult hippocampal neural stem cells by electroporation. Luciferase activity was then determined in co-transfected cells cultured for one day without FGF2, and the value was arbitrarily set at 100%, using Renilla luciferase as an internal control. The NeuroD1-luciferase value was increased 1 day after the induction of neuronal differentiation (RA+FSK, FIG. 2A), but no increase in expression was observed under astrocyte and oligodendrocyte differentiation conditions.

The NeuroD1-luciferase constructs was then utilized to determine the effect of over-expression of several transcriptional regulatory factors. Over-expression of Sox2, CtBP1 and HDAC1 in adult neural stem cells blocked the up-regulation and further repressed NeuroD1-luciferase expression as compared with a control plasmid (FIG. 2A). Introduction of an iRNA specific for the CtBP1 co-repressor gene relieved the repression, demonstrating that the CtBP1 co-repressor protein contributes to repression of the NeuroD1 gene in neural stem cells. In contrast, over-expression of constitutively active β-catenin significantly increased luciferase activity. LEF1 had almost no effect on NeuroD1-promoter activity compared with the control.

HDACs and DNA methylation have been shown to play important roles in gene silencing on chromatin. To determine whether either of these mechanisms contributed to repression of the NeuroD promoter, adult hippocampal neural stem cells were treated with either an HDAC inhibitor, trichostatin A (TSA), or with the de-methylation reagent 5′-aza-cytidine (5AzaC). Total RNA was extracted from cells that had been treated with 5 nM TSA for 2 days and with 3 μM 5AzaC for 4 days. RT PCR analysis was performed with specific primers for NeuroD1 (SEQ ID NOs:7 and 8) and NeuroD2 (SEQ ID NOs:9 and 10) genes and a control gene, GAPDH (SEQ ID NOs:3 and 4). Compared with the level of control mRNA in untreated progenitor cultures, endogenous expression levels of mRNAs of NeuroD1 and NeuroD2 increased in TSA-treated cells with no effects on the expression on GAPDH (FIG. 2B), whereas no activation of these three genes was observed following treatment with 5AzaC, indicating that HDACs play a dominant role in suppressing the NeuroD1 gene in an undifferentiated stage.

To further investigate, in vivo, the physical interactions between these identified proteins and the specific portions of cellular DNAs of NeuroD1 and NeuroD2 genes during neurogenesis, chromatin immunoprecipitation (ChIP) assays were performed. ChIP samples were prepared from neural stem cells (cultured with FGF2) and cells in the neuronal differentiated condition (1 day with RA+FSK), and assayed for protein association on the NeuroD1 and NeuroD2 promoter (FIG. 2C). PCR primers were designed to flank the overlapping binding sequences for the Sox2 and TCF/LEF transcription factors (LEF/Sox overlapping response elements in the NeuroD1 and NeuroD2 promoters (SEQ ID NOs:18 and 19, and 20 and 21, respectively). Input DNA was used to ensure that equivalent amounts of DNA were subjected to CHIP assay and for assessing efficiency of PCR (positive control). No signal was obtained from immunoprecipitation samples when pre-immune rabbit IgG was used (negative control). In stem cells, which expressed high levels of Sox2 protein, both NeuroD1 and NeuroD2 promoters were associated with Sox2 protein, along with CtBP1 and HDAC1 repressor complex (FIG. 2C). In contrast, a de-repressed chromatin states was observed for both NeuroD1 and NeuroD2 genes during neurogenesis, as indicated by a significantly decreased association between the Sox2/CtBP1/IDAC 1 repressor complex with the promoter region. Along with the disappearance of the repressor complex, an increase in the association of LEF1 and Tcf3/4 transcription factors with β-catenin was detected on both promoters (FIG. 2C). Notably, CREB-binding protein (CBP) was also increased on the NeuroD1 and NeuroD2 promoters with TCF/LEF/β-catenin, indicating that an active chromatin state, with transcription of the NeuroD genes, was produced during neurogenesis by a switch from silenced chromatin bound by HDAC1/Sox2/CtBP1 to active chromatin bound by TCF/LEF/β-catenin.

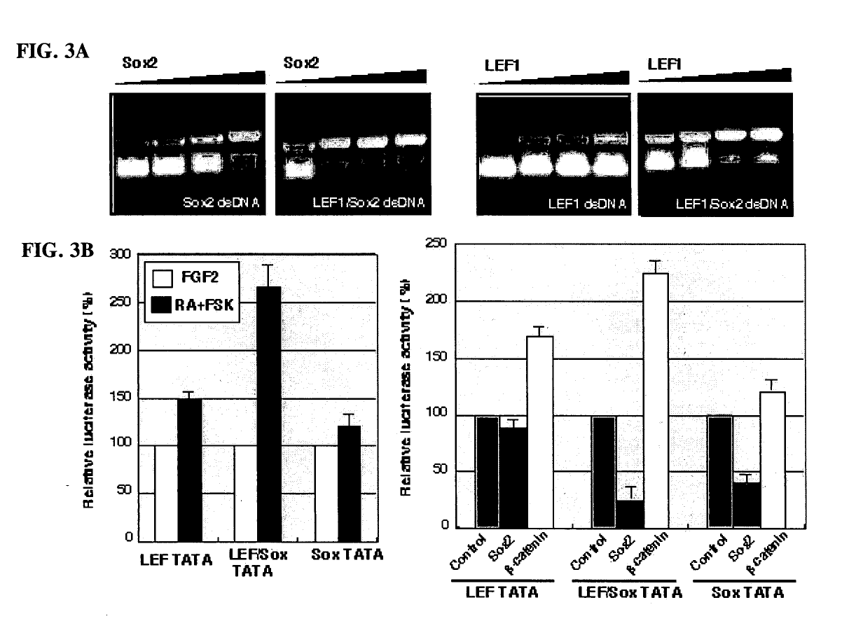

Binding of the DNA elements on the NeuroD promoter by Sox2 or TCF/LEF transcription factors was confirmed by electrophoretic mobility shift assay (EMSA). Double stranded DNA containing only a Sox2 binding site (Sox2 dsDNA) and double stranded DNA containing the LEF/Sox overlapping response element (“LEF1/Sox2” dsDNA) were prepared. A construct that produces Sox2 protein with a flag-tag was introduced in 293T cells, and expressed Sox2 protein was immunoprecipitated with anti-flag antibody. After the purification, the Sox2 protein was incubated with either Sox2 dsDNA or “LEF1/Sox2” dsDNA. As can be seen in FIG. 3A (left panels), Sox2 protein bound both Sox2 dsDNA and “LEF1/Sox2” dsDNA. Interestingly, Sox2 protein showed higher binding activity to “LEF1/Sox2” dsDNA than to Sox2 dsDNA. Similarly, in an EMSA assay using LEF1-flag protein and either LEF1 dsDNA (i.e., dsDNA including only the LEF1 binding sequence) or “LEF1/Sox2” dsDNA, LEF1 protein bound both LEF1 dsDNA and “LEF1/Sox2” dsDNA and exhibited higher affinity to “LEF1/Sox2” dsDNA than LEF1 dsDNA (FIG. 3A, right panels).

### Example 6

### Construction of a Luciferase Reporter Construct Containing LEF/Sox Overlapping Response Elements

A set of reporter constructs useful for investigating transcriptional regulation during the transition from neural stem cells to neuroblasts were prepared. These reporter constructs contain multiple copies of the LEF/Sox overlapping response element. Five copies of the overlapping DNA binding sequence for Sox2 and TCF/LEF were introduced upstream from the ubiquitous 200-bp CMV minimal promoter including a TATA box, and linked to the luciferase gene (LEF/Sox-TATA, FIG. 3B). Five copies of the DNA binding sequence for Sox2 only (Sox-TATA) and five copies of the DNA binding sequence for TCF/LEF only (LEF-TATA) were also fused to the CMV minimal promoter-driven luciferase genes. It should be noted that the minimal promoter region does not contain any Sox or TCF/LEF binding elements. The constructs were introduced into neural stem cells and the luciferase value from transfected cells cultured with FGF2 was arbitrarily set as 100% (control, FIG. 3B, left panel). When LEF/Sox-TATA-luciferase construct was introduced into adult neural stem cells, a significant increase in the luciferase value was detected during neural differentiation (RA+FSK), in comparison with a small increase in expression of the LEF-TATA and Sox-TATA constructs (FIG. 3B, left panel).

As described above, over-expression of Sox2 and β-catenin exhibited characteristic repression and activation of NeuroD1 promoter-mediated expression, respectively. The same results were obtained with reporter constructs including the LEF/Sox overlapping response elements and a minimal promoter. Over-expression of Sox2 protein in neural stem cells resulted in a significant decrease in expression (repression) of LEF/Sox-TATA-luciferase as compared to the control vector (CSC PW; FIG. 3B, right panel). In contrast, over-expression of the constitutively active form of β-catenin resulted in a substantial increase in LEF/Sox-TATA-luciferase expression (FIG. 3B). When the Sox2 binding site was absent from the promoter region (LEF-TATA-luciferase), there was almost no repression observed in response to over-expression of Sox2 protein (FIG. 3B, right panel). β-catenin-dependent up-regulation was observed on the LEF-TATA-luciferase. However, the increase in expression was less than that of the LEF/Sox-TATA-luciferase (FIG. 3B, right panel). Similarly, luciferase gene expression in response to over-expression of β-catenin was significantly decreased on the Sox-TATA-luciferase construct as compared to that of the LEF/Sox-TATA-luciferase construct (FIG. 3B, right panel). These results demonstrate that the overlapping LEF/Sox response elements act as a molecular switch mediating sequential transcriptional regulation from repression to activation during neurogenesis in neural stem cells.

### Example 7

### Repression and Activation of Neural Gene Expression by Sox2 Constitutive Repressor and Sox2 Constitutive Activator

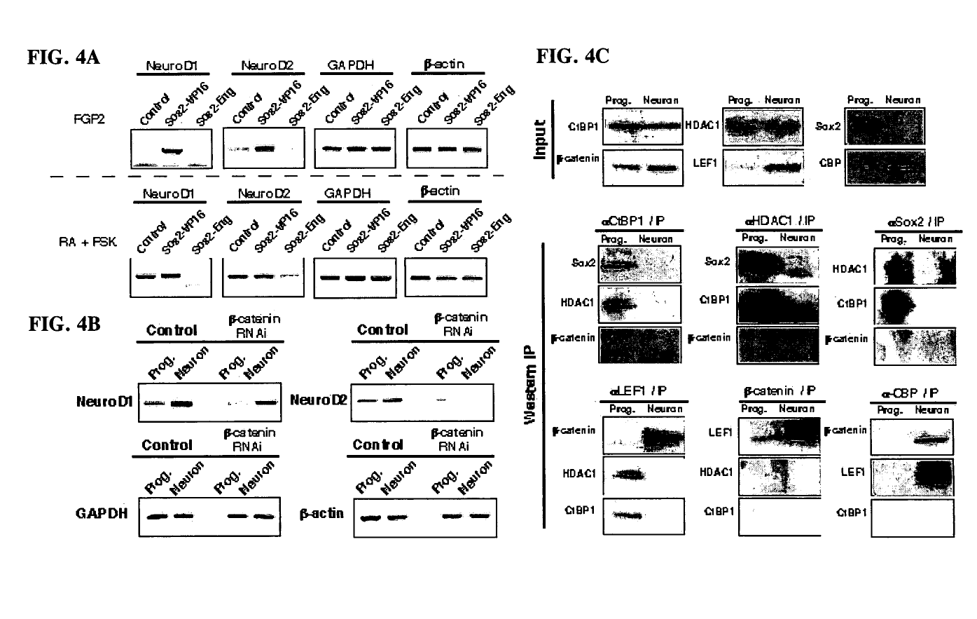

In contrast with previous reports showing that Sox2 protein acts as a transcriptional activator with other protein partners (Nishimoto et al., Mol. Cell. Biol., 19:5453-5465, 1999; Yuan et al., Genes. Dev., 9:2635-2645, 1995; Kamachi et al., Genes Dev. 15:1272-1286, 2001), the results described above demonstrated that the Sox2 protein functions on the NeuroD1 gene as a part of a repressor complex in adult hippocampal neural stem cells. To confirm that the Sox2 protein played a role as a repressor, obligate activator and repressor versions of the Sox2 protein were produced. The HMG domain of Sox2 was fused to the transactivation domain of the cDNA encoding viral protein VP16 to yield the constitutive activator Sox2-VP16 construct. Similarly, the Sox2 HMG domain was fused to the repressor domain of the D. melanogaster Engrailed protein (Sox2-Eng) to produce the constitutive repressor Sox2-Eng. Forced expression of Sox2-VP16 in neural stem cells cultured in the presence of FGF2 increased the expression levels of NeuroD1 and NeuroD2 genes in comparison with controls (FIG. 4A, upper panels). Cells electroporated with Sox2-Eng maintained the suppression of NeuroD1 and NeuroD2 genes in the presence of FGF2 (FIG. 4A, upper panels). Over-expression of Sox2-VP16 in cells cultured under neuronal differentiation conditions (RA+FSK) maintained the up-regulated expression states of NeuroD1 and NeuroD2 genes as compared to controls (FIG. 4A, lower panels). Importantly, cells electroporated with Sox2-Eng did not increase expression of the NeuroD1 and NeuroD2 genes during neuronal induction by RA+FSK (FIG. 4A, lower panels). Neither the GAPDH nor β-actin genes showed any difference in expression levels in response to either the Sox2-VP16 activator or the Sox2-Eng repressor as compared to controls (FIG. 4A), indicating that the Sox2-dependent suppression in neural stem cells was specific to neural specific genes, such as NeuroD1 and NeuroD2. These results demonstrated that Sox2 prevents undifferentiated stem cells from becoming neurons by repressing transcription of NeuroD1 and NeuroD2 genes in adult hippocampal neural stem cells.

### Example 8

### Repression of NeuroD Expression by β-catenin iRNA

The expression of NeuroD1 was up-regulated by the introduction of constitutively active β-catenin in neural stem cells. In contrast, early neuronal stage-specific activation of NeuroD genes is reduced when β-catenin levels are reduced. An siRNA specific for β-catenin was introduced into hippocampal neural stem cells using a lentivirus construct. Down-regulation of NeuroD1 and NeuroD2 was observed in the presence of the β-catenin inhibitory RNA (FIG. 4B). In addition, the increase in expression of the NeuroD genes in response to neuronal induction with RA and FSK was blocked by β-catenin IRNA, indicating an important role for β-catenin in the activation of the NeuroD genes during neurogenesis.

### Example 9

### Interaction of Sox2 with CtBP1 and RDAC1 to Form a Repressor Complex

To elucidate the interactions between transcriptional factors that regulate NeuroD expression during neurogenesis, nuclear extracts from neural stem cells and from differentiating cells were immunoprecipitated with antibodies against Sox2, CtBP1, HDAC1, β-catenin, LEF1 and CBP. Bound proteins were “pulled down” with protein G-conjugated beads and analyzed by Western Blot analysis (FIG. 4C). The immunoblots revealed that Sox2, CtBP1 and HDAC1 interacted directly with each other in neural stem cells. This multi-protein complex no longer appeared when the cells were induced to differentiate into neurons. During neurogenesis, cells actively expressing NeuroD1 and NeuroD2, and produced stable β-catenin in their nuclei (FIG. 4C, upper panel). The nuclear β-catenin bound tightly with LEF 1 and CBP proteins to form a multi-protein complex. Although the LEF 1 protein was also capable of binding HDAC1 and CtBP1, the binding affinity was very weak compared to LEF1 binding with β-catenin and CBP.

### Example 10

### Correlation between the Timing of the Up-Regulation of smRNA, NeuroD1, and Retrotransposon LINE Genes during Adult Hippocampal Neurogenesis

To investigate the regulatory mechanism of production of smRNA, the exact timing of the appearance of NRSE smRNA was assessed by Northern blotting. Nuclear fractions were collected from adult hippocampal neural stem cells at various time points during neuronal differentiation, and the purified RNAs were subjected to analysis. Total RNA was extracted with TRIzol reagent (Gibco-BRL). To prepare the cytoplasmic fraction, cells were incubated in digitonin lysis buffer (50 mM HEPES/KOH, pH 7.5, 50 mM potassium acetate, 8 mM MgC12, 2 mM EGTA and 50 μg/mL digitonin) on ice for 10 min. The lysate was centrifuged at 1,000×g and the supernatant was collected as the cytoplasmic fraction. The pellets resuspended in NP-40 buffer (20 mM Tris-HCl, pH 7.5, 50 mM KCl, 10 mM NaCl, 1 mM EDTA and 1% NP-40) were used as the nuclear fraction. Purified RNA was loaded on a 3.5% NuSieve-Seakem™ agarose gel (FMC Inc.) and transferred to a Hybond-N™ nylon membrane (Amersham Co.). The membrane was probed with synthetic oligonucleotides that were complementary to the sequences of each sNRSE or asNRSE that had been labeled with ³²p by T4 polynucleotide kinase (NEB). Pre-hybridization and hybridization were carried out using EazyHyb solution (Clontech) following manufacturer's instructions.

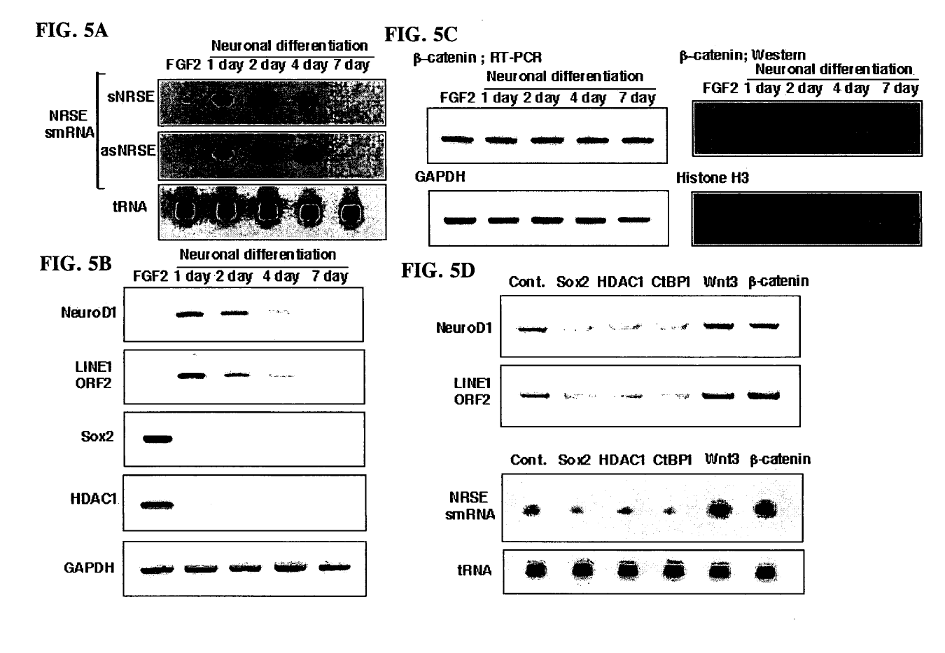

Both sense strand NRSE RNA (sNRSE smRNA) and antisense NRSE RNA (asNRSE smRNA), corresponding to about 20 nt in length, appeared at early stages of neurogenesis in cells that were treated with 1 μM retinoic acid (RA) and 5 μM forskolin (FSK) for 1-4 days (FIG. 5A). Cells cultured with RA+FSK for 7 days did not express the smRNA. Cells at post-1 day of neuronal induction contained the highest amount of smRNA, and the RNA levels gradually decreased during neuronal differentiation.

To compare the timing of the up-regulation of both of the critical neuronal inducers, NeuroD1 and NRSE smRNA, the expression levels of the NeuroD1 gene and of several other genes related to the regulation of NeuroD1 were examined by RT-PCR at the same time points during neuronal differentiation. As can be seen in FIG. 5B, NeuroD1 expression was up-regulated to its highest levels by 1 day after neuronal induction (RA+FSK) and was gradually down-regulated as differentiation progressed, similar to expression of smRNA (FIG. 5A).

NRSE sequences, including a variety of nucleotide modifications, are interspersed in the genome at more than 1,800 sites (Bruce et al., Proc. Natl. Acad. Sci. USA, 101(28):10458-63, 2004). Retrotransposon elements are also dispersed throughout in the genome and can influence the expression of nearby genes (Speek, Mol. Cell. Biol., 21:1973-1985, 2001; Nigumann et al., Genomics, 79:628-634, 2002; Kashkush et al., Nature Genet., 33:102-106, 2003). To determine the relationship between NRSE sequences and retrotransposon elements, the expression state of retrotransposon genes was assessed at times when NRSE smRNAs actively function as RNA transcriptional modulators. As can be seen from FIG. 5B, a transient up-regulation of ORF2, an open reading frame encoded by LINE-1 that has both endonuclease and reverse transcriptase activity, was observed at the same time during early neurogenesis that high levels of smRNA and NeuroD1 expression are observed (FIG. 5B).

### Example 11

### Transcriptional Regulation of NRSE smRNAs in Neural Stem Cells

As discussed above, activation of the NeuroD1 gene to direct neuronal differentiation in adult hippocampal neural stem cells depended on the disappearance of the repressor complex containing Sox2 and was mediated by an activator complex including β-catenin. The expression of Sox2 and HDAC1 genes, which repress the NeuroD1 gene in neural stem cells, was dramatically reduced upon neuronal induction, with similar timing (FIG. 5B). Expression of β-catenin was also evaluated at the same time points used for smRNA analysis during neuronal differentiation. The RNA and nuclear fractions of protein were analyzed by RT-PCR and Western blotting (FIG. 5C). FIG. 5C shows that the nuclear accumulation of β-catenin protein was highest one day after neuronal induction (FIG. 5C, right panels), at the same time point as the up-regulation of smRNA and NeuroD1 (FIGS. 5A and 5B), even though the RNA expression levels were unchanged during neuronal differentiation (FIG. 5C, left panels). These data demonatrated the coordinated transcriptional regulation between smRNA, NeuroD1, and retrotranposon LINE.

To determine whether the NRSE smRNA was regulated in a manner comparable to that of NeuroD1, the effects of genes involved in NeuroD1 regulation on NRSE smRNA expression were examined. Sox2, CtBP1, HDAC1, Wnt3 and β-catenin-expressing lentiviral vectors were constructed using CSC PW, a lentiviral vector, and the cDNA was first amplified/cloned by PCR. Each expression cassette was sub-cloned at the 3′ end of the CMV promoter on CSC PW using restriction enzyme sites of BamH1 and Pme I. The production of lentivirus has been described elsewhere (Pfeifer et al., Proc. Natl. Acad. Sci. USA, 98:11450-11455, 2001), and infections were almost 100% (viral titers were >1.5×104 Tu/ng defined by the P24 assay). Murine NeuroD1 promoter was cloned by PCR from genomic DNA and inserted into CSC PW-Luci at the site of the CMV promoter using the restriction enzyme sites of Cla I and Bam HI. Expression constructs for Sox2, HDAC1, CtBP1, Wnt3, and constitutively active β-catenin were electroporated into adult hippocampal neural stem cells, and the cells were cultured in RA+FSK for two days after incubation with FGF2 for one day. The extracted RNAs were examined using RT-PCR and Northern blotting analyses (FIG. 5D). The over-expression of Sox2, HDAC 0 and CtBP1 blocked activation and repressed expression of the LINE1 ORF2 transcript and the NeuroD1 gene during neuronal differentiation (FIG. 5D; upper panels). Importantly, forced expression of Sox2, HDAC1 and CtBP1 resulted in resistance to the neuronal lineage-specific activation of NRSE smRNA (FIG. 5D, lower panels). Conversely, over-expression of Wnt3 and constitutively active β-catenin further activated the expression of ORF2 LINE1 and NeuroD1 genes, as compared to controls (FIG. 5D; upper panels). In addition, NRSE smRNA was up-regulated by the over-expression of Wnt3 and constitutively active β-catenin. β-catenin and Wnt3 activated smRNA and retrotransposon LINE as well as NeuroD1 expression, indicating that an extracellular Wnt factor acts as a neuronal enhancer in adult hippocampus. These results showed that the transcription of NeuroD1 and LINE1 genes and the production of NRSE smRNAs actively occur at the same time during early neurogenesis, and are under the influence of the same transcriptional regulation system.

### Example 12

### Existence of DNA Elements Responding to Neuronal Lineage-Specific Induction of NeuroD1 Promoter and Retrotransposon LINE1 Gene

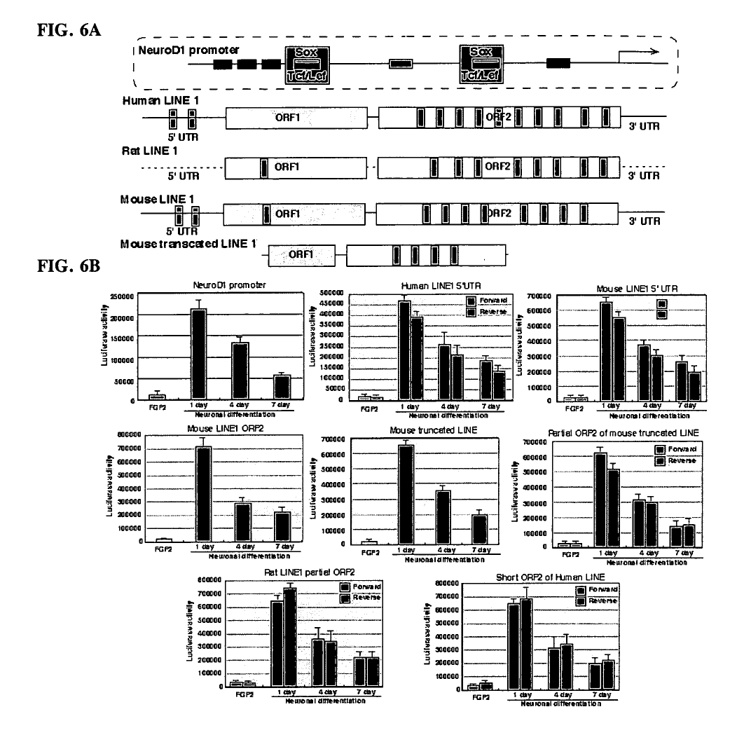

As described above, the LEF/Sox response element regulates expression of NeuroD1 in adult hippocampal neuroblast cells. The overlapping DNA sequence for Sox protein and TCF/LEF proteins controls repression in adult neural stem cells by binding with Sox2/CtBP1/HDAC1 complex. In contrast, activation in hippocampal neuroblasts is controlled by binding with TCF/LEF/β-catenin/CBP complex. As discussed above, the expression of NeuroD1, LINE1 and NRSE smRNA are under a common transcriptional control. Therefore, the promoter regions of these genes were analyzed, focusing on the LEF/Sox response element. FIG. 6A shows overlapping LEF/Sox response elements in the NeuroD1 promoter (top, dotted panel). Human LINE (M80343, ˜6 kb), mouse LINE (Ml3002, 7.7 kb), and a truncated mouse LINE sequence (X03725, ˜2.7 kb) were analyzed, and all were found to contain numerous LEF/Sox response elements, (12, 10 and 4 LEF/Sox response elements, respectively; FIG. 6A). The sequence for rat LINE1 (X03095) was also analyzed and found to contain 8 LEF/Sox sequences.

The transcriptional activity of LINE elements was analyzed in tandem with the murine NeuroD1 promoter in neural stem cells (cultured with FGF2) and during neuronal differentiation (induced with RA+FSK). The NeuroD1 promoter, 5′ UTR regions of human and mouse LINE, mouse LINE1 ORF2, mouse truncated LINE, and partial ORF2 from human, mouse and rat LINE1 were each cloned and operably linked to the luciferase gene. FIG. 6A illustrates that they all carry multiple LEF/Sox DNA response elements. The LINE-luciferase constructs were introduced into adult hippocampal neural stem cells by electroporation, and the Renilla luciferase construct was co-transfected as an internal control. Luciferase activity was measured with Dual-Luciferase™ Reporter Assay System (Promega) according to the manufacturer's protocol. The luminescent signal was quantified with a luminometer (Lumant LB 9501). As an internal control, a plasmid containing Renilla luciferase gene was co-transfected.

The NeuroD1-luciferase value was increased more than 10 times one day after the induction of neuronal differentiation, and the level gradually decreased as differentiation progressed (4 days and 7 days; FIG. 6B, upper left), consistent with the expression profile of endogenous NeuroD1. As shown in FIG. 6B, the 5′ untranslated regions (UTRs) of human and mouse LINE exhibited promoter activity in both orientations and were up-regulated by neuronal induction (FIG. 6B, upper right graph). The 5′ UTR region as well as the LINE sequence itself possessed promoter activity, even in the truncated form (FIG. 6B). Furthermore, the ORF2 region of LINE1 and partial ORF2 fragments from mouse (1.5 kb), rat (1 kb) and human (0.5 kb) LINE1 all showed promoter activity (FIG. 6B). These partial ORF2 fragments were abundantly embedded in each genome. The expression was significantly activated more than 50-fold by neuronal induction, and the level gradually decreased as differentiation progressed, similar to NeuroD1. These correlations demonstrated that the same transcriptional factors (the transition of TCF/LEF/β-catenin/CBP activator complex from Sox2/CtBP1/HDAC1 repressor complex) regulate expression of NeuroD1 and LINE through the LEF/Sox response element during early stages of neurogenesis.

### Example 13

### Genomic Distribution of NRSE Sequence and Retrotransposon LINE

A computer search of LINE elements was conducted to determine their distribution in mouse genome. The location of NRSE loci in the mouse genome was also determined. Genomic locations of LINE elements in mouse and rat were obtained using the RepeatMasked annotations provided on the UCSC public genome database (http://genome.ucsc.edu). Perl-script code was written to find loci of all perturbations of the NRSE consensus sequence (nnCAGCACCnnGGACAGnnnC: SEQ ID NO: 17) in the mouse (mm5) and rat (m3) genomes downloaded from the UCSC public database. NRSE loci with a non-trivial LINE element (SW Score>2500) within 10,000 bases were located. NRSE loci with nearby LINE elements on both sides were further considered, and sequence for several examples was downloaded from the UCSC browser and used as primers in RT analysis. Celera Discovery System genomes (http://www.celera.com) for mouse, rat and human were also used to validate the proximal presence of NRSE elements and LINE elements. NRSE and LINE sequences (known to contain the LEF/Sox DNA regulatory element) were found in the Celera genomes using the Celera blast function, and sequences surrounding interesting NRSE elements were downloaded for quantitative PCR. Globally, NRSE locations were similar between the two providers (Public versus Celera).

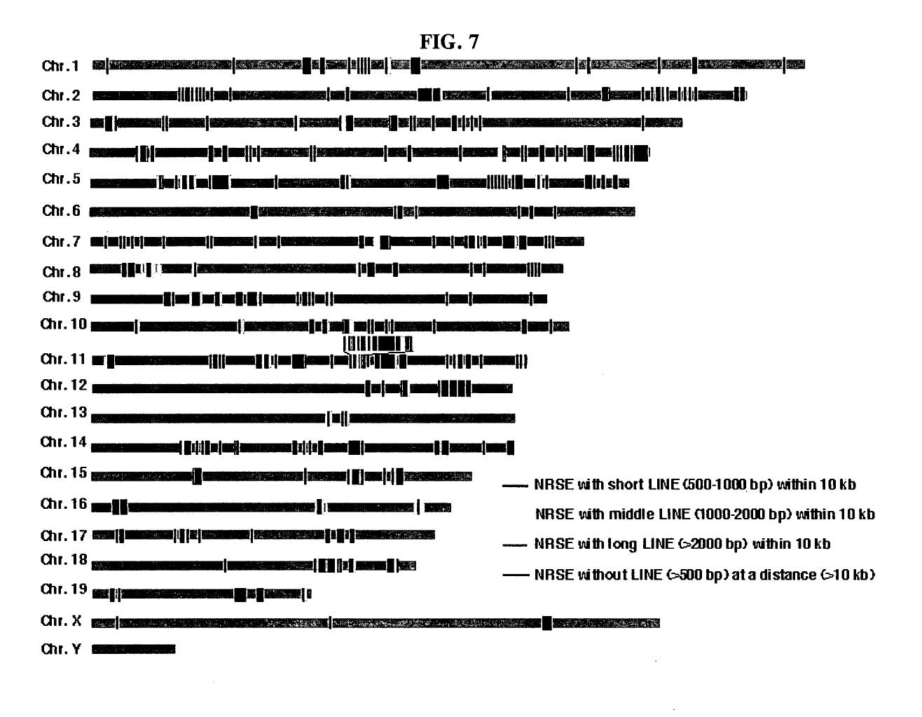

Recently, it was reported that more than 1,500 variations of the NRSE sequence were coded in the mouse genome and more than 1,800 in the human genome (Bruce et al., Proc. Natl. Acad. Sci. USA, 101(28):10458-63, 2004). Since variants of the NRSE consensus sequence can be recognized by the NRSF/REST transcriptional factor (Chong et al., Cell, 80:949-957, 1995; Schoenherr and Anderson, Science, 267:1360-1363, 1995; Schoenherr et al., Proc. Natl. Acad. Sci. USA, 93:9881-9886, 1996; Chen et al., Nat. Genet., 20:136-142, 1998; Palm et al., J. Neurosci., 18:1280-1296, 1998; Huang et al., Nat. Neurosci., 2:867-72, 1999, Bruce et al., Proc. Natl. Acad. Sci. USA, 101(28): 10458-63, 2004), such variants were also included in the search. Using public and Celera databases, approximately 330 of a possible 1,024 combinations of NRSE sequences exist in the mouse genome. FIG. 7 shows the wide distribution of these NRSE sequences in the mouse genome and their relationship to nearby LINE elements (within 10 kb). Since there are thousands of partial LINE elements embedded in the genome, almost all NRSEs were near the LINEs if short, truncated LINE fragments (less than 500 bp) were included in the search. Since these shorter fragments have not been shown to have promoter activity, they were excluded from this analysis. The LINEs that are located within 10 kb of a NRSE sequence are shown, and include “short” (500-1000 bp), “mid-length” (1000-2000 bp) and “long” (greater than 2000 bp) (FIG. 7). Most chromosomes, other than the Y chromosome, contained adjacent NRSE and LINE sequences. In several cases a NRSE sequence was surrounded by LINE sequences on both sides. These close associations between NRSE and LINEs suggest that genomic NRSE sequences were transcribed from embedded LINE elements, which function as inherent promoters. This co-localization of NRSE and LINE sequences was also seen in the rat and human genomes.

### Example 14

### Early Neuronal Up-Regulation of NRSE Transcription from LINE Inherent Promoters

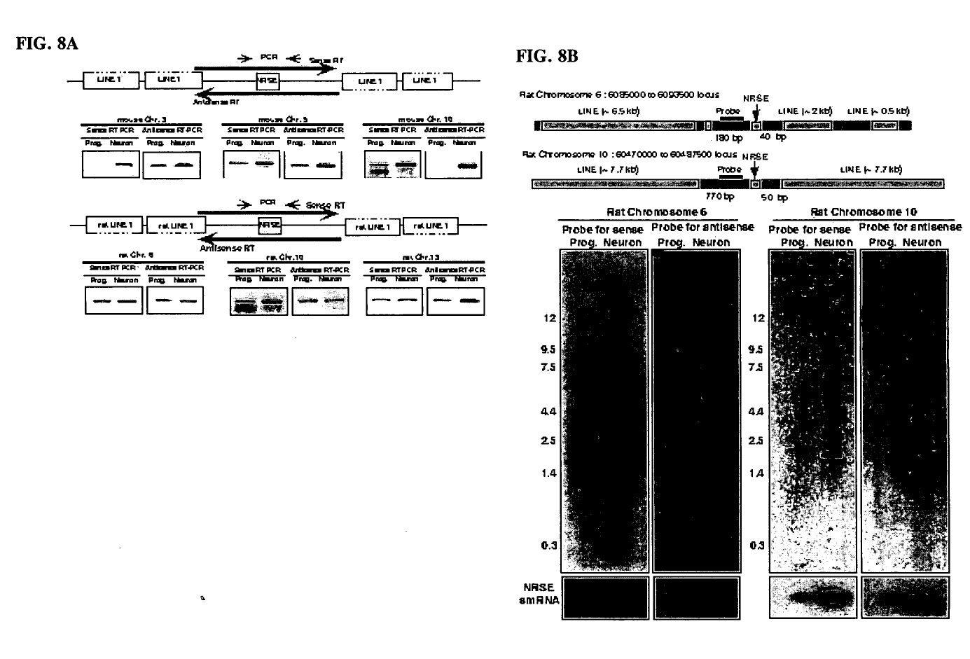

Most LINEs are embedded in the genome as repetitive sequences. Genomic analysis revealed that some scattered LINE regions included NRSE sequences within the cluster, in which NRSE sequences were flanked or surrounded by multiple LINEs on both sides (LINE-NRSE-LINE). To determine whether endogenous genomic RNA transcripts containing NRSE could be generated from a nearby LINE element as promoter, specific primers were designed for reverse transcription (RT) to hybridize with sense or antisense RNA transcripts containing the NRSE sequence (FIG. 8A). Three exemplary sites were selected for analysis from among the examples of close localization between LINE and NRSE in the mouse genome. Transcription was examined at sites on chromosomes 3, 5, and 10 in adult murine neural progenitor cells during neurogenesis. To detect RNA transcripts that corresponded to the LINE-NRSE-LINE loci, primers for RT were designed to specifically hybridize to sense as well as antisense RNA strands, and PCR primers were used to amplify the corresponding antisense and sense DNA strands. As shown in FIG. 8A, the RNA was transcribed in both directions, and the transcription was highly activated during the early stages of neurogenesis. LINE and NRSE sequences throughout the rat genome were also evaluated to determine the consistency of the close spatial relationship between them. As was found in the mouse, several sites of scattered NRSE sequences flanked by LINE regions (LINE-NRSE-LINE) were detected in the rat genome. Three exemplary sites, located on rat chromosome 6, 10 and 13, were examined by RT-PCR analysis (FIG. 8A). The expression of RNAs at the LINE-NRSE-LINE site was detected for both sense and antisense orientation, and the transcription from LINEs was much higher in cells during neurogenesis than in neural stem cells (FIG. 8A, lower panels).

### Example 15

### NRSE smRNA Precursors are Produced from Scattered LINEs during Early Neurogenesis

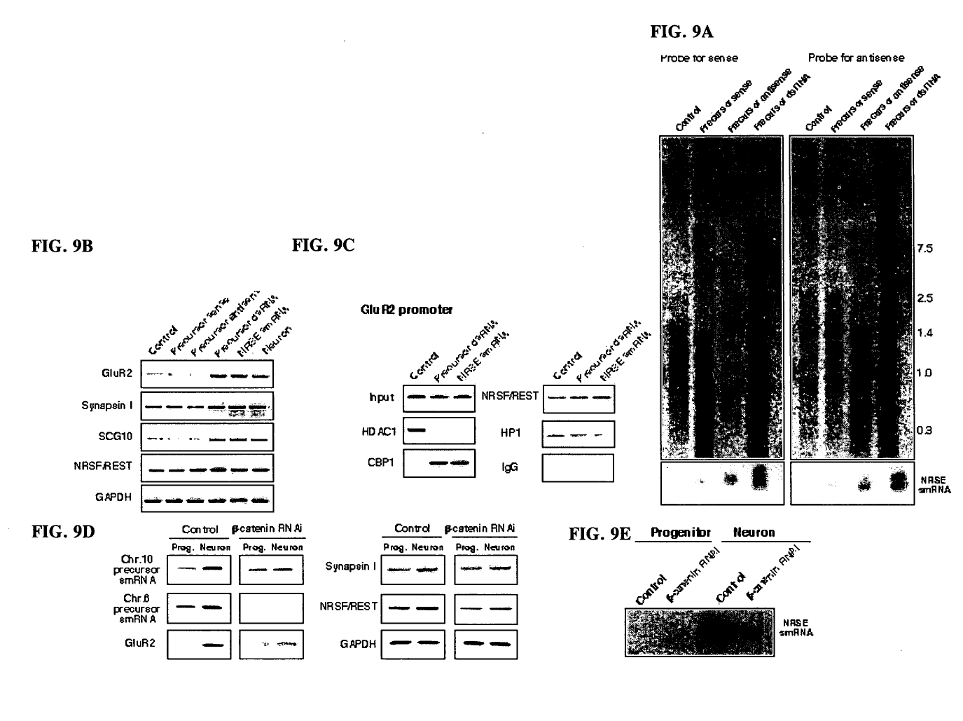

In situ hybridization was performed to detect NRSE RNA transcripts generated from LINE elements as promoters in adult rat hippocampal cells. Immunostaining for beta-tubulin III protein (TUJ1) was conducted simultaneously as a control. The probe for in situ hybridization was designed to hybridize to sequences adjacent to the NRSE site on the sense or antisense RNA transcripts from LINEs (LINE-NRSE) on rat chromosome 10. As shown in FIG. 9C, DAPI (blue) and sense LINE-NRSE RNA (green) co-localized in the nucleus of cells during neurogenesis (RA+FSK for two days, upper right panel), whereas the expression of sense LINE-NRSE RNA was not detected at significant levels in cells at the progenitor stage (FGF2, upper left panels). Similar observations were seen for antisense LINE-NRSE RNAs (FIG. 9C, lower panels). C) In situ hybridization against the RNA transcript containing NRSE sequences generated from LINE elements as promoters in adult rat hippocampal cell. TUJ 1-positive cells during neuronal differentiation expressed higher levels of LINE-NRSE RNAs, consistent with the observation from RT-PCR studies (FIG. 8A).

To determine the sizes of RNA transcripts transcribed using promoter elements in LINEs, Northem Blot analysis was conducted using extracted RNA from cells at an undifferentiated stage and at an early stage of neurogenesis. Genomic loci on rat chromosome 6 and 10 were assessed as typical examples for the analysis. The probe was designed to hybridize to the flanking region between LINE and NRSE on LINE-NRSE-LINE RNAs (FIG. 8B). The LINE-NRSE-LINE site on chromosome 6 (located between bases 6085000 to 6093500 on UCSC rn3 genome) has one full length LINE (6.5 kb) and one truncated LINE (2 kb). The truncated 2 kb sequence includes a partial ORF1 and ORF2 and 3 LEF/Sox response elements. The NRSE sequence is surrounded by these LINEs (separated by 180 bp and 40 bp, respectively). The LINE-NRSE-LINE site on chromosome 10 (base position 60470000 to 60487500) has two nearly full length LINEs (7.7 kb), and the NRSE sequence is surrounded by these LINEs at a distance of 770 bp and 50 bp, respectively. As shown in FIG. 8B, long transcripts were produced in both orientations (sense and antisense LINE-NRSE-LINE) at both chromosome 6 and 10 loci. Several different sizes of RNAs were detected, indicating that RNA transcription can be initiated at several sites and/or that multiple termination sites exist. The RNA production level was higher at the early stage of neurogenesis than at the undifferentiated stage in all cases. Neurogenesis stage-specific NRSE RNA expression using adjacent LINEs as promoters was also detected on rat chromosomes 3 and 13.

### Example 16

### Neuronal Differentiation Mediated by the NRSE smRNA

To examine the effect of NRSE RNA transcripts generated from flanking LINE sequences on neuronal differentiation, an approximately 1 kb sequence including the non-coding LINE-NRSE-LINE sequence on rat chromosome 10 was subcloned under the regulatory control of the CMV promoter into a lentivirus vector in both sense and antisense orientations. Control mock virus infection and the over-expression of each single sense or antisense strand (precursor sense and precursor antisense) had almost no effect on hippocampal neural progenitor cells. However, when both complementary strands were expressed simultaneously, resulting in the formation of dsRNA (precursor dsRNA), the morphology of the neural progenitor cells dramatically changed two days after the expression was initiated. The transfected cells were stained with neuronal lineage-specific antibodies (TUJ1, Map2AB, NF200, Calbindin and Synapsin I) and were negative for glial markers (GFAP and RIP). Both sense and antisense RNA transcripts were detected in Northern blotting analysis, together with several longer transcripts that are likely poly A-tailed RNA transcripts of the induced RNA and/or other non-coding RNAs generated at the same chromosome locus as the indirect result of neuronal differentiation by the induction of precursor dsRNA. Importantly, the induction of both sense and antisense long precursor RNAs increased the amount of short NRSE smRNAs in the presence of FGF2 (FIG. 9A), indicating that these long transcripts with NRSE sequence in both directions function as precursor RNAs in the generation of NRSE smRNA. The expression of neuronal genes that have NRSE regulatory sequences in their promoter regions (Synapsin I, SCG10 and GluR2) was highly up-regulated by expression of the precursor dsRNA, as well as NRSE smRNA induction (FIG. 9B). This activation event was also confirmed by chromatin immunoprecipitation (CHIP) for the NRSE site on the GluR2 promoter region (FIG. 9C). ChIP assay was done essentially following the manufacturer's protocol using a ChIP assay kit (Upstate). RT-PCR was performed using total RNA extracted from HCN A94 cells. One (1) μg RNA was used for first-strand cDNA synthesis with SuperScript II (GibcoBRL). HDAC1 and HP1 were found to associate with the NRSE site to create silenced heterochromatin in neural stem cells, whereas expression of precursor dsRNA that generated NRSE smRNA led to de-repression with disappearance of these silencing factors and further activation of CBP1 on the locus. In both cases, NRSF/REST tightly associated with the NRSE site, indicating that the appearance of NRSE smRNA, from precursor dsRNA, is the key step in the transition of NRSF/REST from a repressor to an activator.

### Example 17

### Promoters of Genome-embedded LINEs are Co-opted to Produce NRSE smRNAs through the Generation of Precursor dsRNA

As shown above, NeuroD1 promoter activity is regulated by a molecular switch mediated by LEF/Sox transcriptional factors during early neuronal differentiation. To determine whether NRSE RNAs were also regulated by this molecular switch, β-catenin expression was reduced using a siRNA. A lentiviral construct that expressed siRNAs for β-catenin was introduced into cells. RT-PCR analysis was performed using RNA extracts from cells infected with control lentivirus and the β-catenin siRNA lentivirus. As shown in FIG. 9D, the levels of precursor RNAs for NRSE smRNA (precursor smRNA) on rat chroromosomes 10 and 6 were decreased, resulting in the down-regulation of NRSE-containing genes (GluR2 and Synapsin I). The same RNAs were also subjected to Northern blotting analysis, which revealed that the amount of NRSE smRNA was decreased by the β-catenin siRNA.

### Example 18

### Chromatin Structure at NRSE Sites Surrounded by LINEs

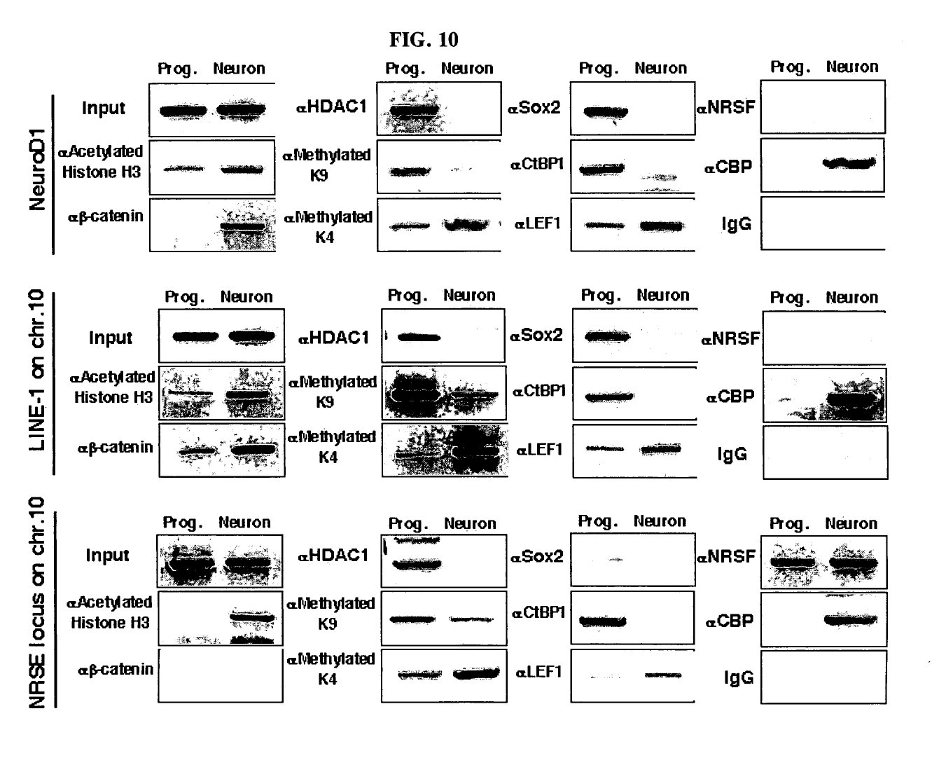

To determine the in vivo chromatin structure and the localization of the transcriptional complex regulating the RNA transcription at the LINE-NRSE-LINE locus, ChIP analysis was performed on extracts from neural stem cells and from cells during neuronal differentiation. Purified DNA fragments from fixed whole cells extracts (input) or co-precipitated with specific antibodies were amplified by PCR using primers (SEQ ID NOs:30 and 31) that surround NRSE sites in the LINE-NRSE-LINE locus on rat chromosome 10. PCR primers surrounding LEF/Sox DNA regulatory element in the NeuroD1 promoter and for the ORF2 region of LINE1 were prepared to determine the nature of chromatin states of promoters for the NeuroD1 gene and for the precursor RNA of NRSE smRNA. In neural stem cells, HDAC1 is associated with NRSE sites in the LINE-NRSE-LINE locus, in addition to sites within the NeuroD1 promoter and ORF2 on LINE1 (FIG. 10). The association of the CtBP1 co-repressor, together with Sox2, on LEF/Sox response elements on both NeuroD1 promoter and LINE1 suggests that the repressor complex of Sox2/HDAC1/CtBP1 represses downstream genes in neural stem cells. At the NRSE site on the LINE-NRSE-LINE locus, NRSF/REST was associated in both neural stem cells and cells undergoing neuronal differentiation, reflecting the unique property of specific recognition of NRSF/REST on the NRSE DNA sequence as a bipotent transcription factor regulated by the presence of NRSE smRNA (as previously described in Kuwabara et al., Cell, 116:779-793, 2004). CtBP1 bound the NRSE site on the LINE-NRSE-LINE locus in neural stem cells, demonstrating that it functions as a co-repressor with NRSF/REST and HDAC 1. This result is consistent with previous findings that CtBP1 associated with NRSF/REST through CoREST (Shi et al., Nature, 422:735-738, 2003).

Histone modification was evaluated to determine the nature of chromatin at the NRSE site on the LINE-NRSE-LINE locus and the LEF/Sox response elements in the NeuroD1 promoter and in LINE1. Accumulation of Histone H3 lysine-9 methylation (diMeK9-H3), Histone H3 lysine-4 methylation (diMeK4-H3) and Histone H3 acetylation (Ac-H3) was assessed. As shown in FIG. 10, diMeK9-H3 associated with HDAC1 on all three loci in neural stem cells, indicating their repressed chromatin structure. In contrast, diMeK4-H3 accumulated highly on the NeuroD1 promoter, LINE1 and NRSE site, together with Ac-H3 and CBP with HAT activity, corresponding to an active transcriptional state of these three genes during early neurogenesis. The accumulation of β-catenin and LEF1 on LEF/Sox response elements in the NeuroD1 promoter and LINE1 during neurogenesis indicated that the repressor complex of Sox2/HDAC1/CtBP1 is replaced by the TCF/LEF/β-catenin/CBP complex to activate transcription.

### Example 19

### Expression of Precursor RNA of NRSE smRNA in the Adult Hippocampus

Expression of precursor RNAs (that is, RNAs including NRSE sequence generated from nearby LINE1 at the LINE-NRSE-LINE locus) and NRSE smRNA in the dentate gyrus of the adult hippocampus was analyzed by in situ hybridization. Immunostaining of NeuroD1 and with the mature neuronal marker, NeuN, was also performed. Cells were fixed in fix/permeablization buffer (50 mM HEPES/KOH, pH 7.5, 50 mM potassium acetate, 8 mM MgC12, 2 mM EGTA, 2% paraformaldehyde, 0.1% NP-40, 0.02% SDS) for 15 min. The FITC-/Rhodamine-labeled oligodeoxynucleotide probes matching complementary to asNRSE and NRSF/REST mRNA were denatured for 10 min at 70° C. and chilled. Hybridization buffer, containing 20% dextran sulfate and 2% BSA in 4×SSC, with probes were placed on the cells for 16 h. Cells were rinsed in 2×SSC/50% formamide and in 2×SSC for 20 min each.

Immunofluorescence studies were performed basically as described (Gage et al., Proc. Natl. Acad. Sci. USA, 92:11879-11883, 1995) with the following antibodies: rabbit anti-beta tubulin-III (TUJ1; 1/7500, Covance), guinea pig anti-GFAP (1:500; Advanced Immunochemical, Inc), rabbit anti-NF200 (Advanced Immunochemical, Inc), mouse anti-RIP (1/250, Immuno), rabbit anti-Calbindin (Advanced Immunochemical, Inc) and DAPI (Sigma). All secondary antibodies were from Jackson ImmunoResearch. Images were analyzed using the Bio-Rad Radiance confocal imaging system (Hercules, Calif.).

Cells expressing NeuroD1 were only detected at the inner layer of the rat hippocampal dentate gyrus, where adult neurogenesis is continuously occurring (van Praag et al., Nature, 415:1030-1034, 2002; Kempermann et al., Development, 130:391-399, 2003). These cells were negative for the mature neuron marker NeuN.

The in situ probes for the precursor RNA for NRSE smRNA were designed to hybridize the flanking sequence near the NRSE site in LINE-NRSE-LINE locus on chromosome 10. The NRSE smRNA was expressed in the subgranular inner layer region of the dentate gyrus, as was NeuroD1. The RNA product from the genome-embedded LINE promoter, which included the NRSE sequence in sense orientation (precursor sense), was expressed in similar pre-neuronal areas in the adult hippocampus. The precursor RNA in antisense orientation of the same genomic locus of LINE-NRSE-LINE was also restricted to the inner layer of the dentate gyrus. These data were consistent with the in vitro data and demonstrated that the precursor RNA for NRSE smRNA was generated by both directional transcriptions from LINE1 at an early stage in neuronal differentiation in the adult hippocampus.

While this disclosure has been described with an emphasis upon particular embodiments, it will be obvious to those of ordinary skill in the art that variations of the particular embodiments may be used and it is intended that the disclosure may be practiced otherwise than as specifically described herein. Accordingly, this disclosure includes all modifications encompassed within the spirit and scope of the disclosure as defined by the following claims:

We claim:

1. A method of identifying an agent that regulates differentiation of a stem cell into a neural lineage cell, the method comprising:
(a) contacting with at least one agent a cell, which cell comprises a polynucleotide that encodes a reporter operably linked to a transcription control sequence, wherein the transcription control sequence comprises one or more LEF/Sox overlapping response elements; and,
(b) detecting a relative change in expression of the reporter, wherein the change in expression is measured in comparison to a control cell, which control cell is not contacted with the agent, thereby identifying the agent that regulates differentiation of the stem cell into a neural lineage cell.

2. The method of claim 1, wherein the cell contacted with the agent and the control cell are stem cells or neural lineage cells.

3. The method of claim 1, comprising detecting a relative increase in expression of the reporter as compared to the control cell, thereby identifying an agent that induces differentiation of the stem cell into a neural lineage cell.

4. The method of claim 1, comprising detecting a relative decrease in expression of the reporter as compared to the control cell following exposure of the stem cell and the control cell to a stimulus, which stimulus produces an increase in expression of the reporter in the absence of the agent, thereby identifying an agent that inhibits differentiation of the stem cell into a neural lineage cell.

5. The method of claim 4, wherein the relative decrease comprises a constant level of expression of the reporter as compared to an increase in expression of a reporter in the control cell.

6. The method of claim 4, wherein the stimulus comprises at least one of retinoic acid (RA) and forskolin (FSK).

7. The method of claim 4 wherein the exposure to the stimulus comprises expressing at least one polynucleotide encoding a binding factor selected from the group consisting of β-catenin, a Lef transcription factor, a Tcf transcription factor, CREB-binding protein (CBP), glycogen synthase kinase (GSK3), and a wnt activator in the cell and the control cell.

8. The method of claim 1, wherein the reporter is an optically detectable reporter or a selectable marker.

9. The method of claim 1, wherein each of a plurality of stem cells is contacted with at least one member of a composition library.

10. A method of identifying an agent that regulates differentiation of a stem cell into a neural lineage cell, the method comprising:
contacting a nucleic acid comprising a polynucleotide sequence comprising one or more LEF/Sox overlapping response elements with a reaction mixture comprising
at least one binding factor capable of specific binding to the LEF/Sox overlapping response element; and
at least one agent; and
detecting a change in binding of a component of the reaction mixture to the nucleic acid, thereby identifying an agent that regulates differentiation of a stem cell into a neural lineage cell.

11. The method of claim 10, wherein the reaction mixture comprises a soluble extract of a cell.

12. The method of claim 10, comprising detecting a change in binding of a component of the reaction mixture to the nucleic acid comprises detecting binding to the nucleic acid of one or more of β-catenin, Lef1, Tcf3, Tcf4, CBP and Sox2.

13. The method of claim 10, comprising detecting a change in binding of the agent to the nucleic acid.

14. The method of claim 10, comprising detecting binding of the component of the reaction mixture by detecting a mobility shift of the polynucleotide comprising the LEF/Sox overlapping response elements.

15. A method of modulating differentiation of a stem cell into a neural lineage cell, the method comprising expressing in a stem cell at least one polypeptide that binds to a LEF/Sox overlapping response element, wherein binding of the polypeptide induces modulates differentiation of the stem cell into a neural lineage cell.

16. The method of claim 15, wherein expressing the at least one polypeptide that binds to a LEF/Sox overlapping response element comprises introducing into the stem cell a nucleic acid comprising a polynucleotide sequence that encodes the polypeptide operably linked to a promoter.

17. The method of claim 15, wherein binding of the at least one polypeptide to the LEF/Sox overlapping response element induces the stem cell to differentiate into a neural lineage cell.

18. The method of claim 15, wherein binding of the at least one polypeptide to the LEF/Sox overlapping response element inhibits differentiation of the stem cell into a neural lineage cell following exposure to a stimulus that induces differentiation in the absence of the polypeptide.

19. A recombinant nucleic acid comprising a heterologous polynucleotide sequence operably linked to a transcription control sequence, which transcription control sequence comprises one or more LEF/Sox overlapping response elements.

20. The recombinant nucleic acid of claim 19, further comprising at least one promoter.

21. The recombinant nucleic acid of claim 19, wherein the heterologous polynucleotide sequence encodes a polypeptide or an RNA.

22. A method of expressing a polynucleotide sequence in a cell, the method comprising:
introducing into a cell the recombinant nucleic acid of claim 19; and,
growing the cell under conditions in which TCF/LEF binds to the overlapping response elements, thereby expressing the polynucleotide sequence in the cell or at least one progeny thereof.

23. The method of claim 22, comprising introducing the nucleic acid into a cell comprising at least one additional heterologous nucleic acid, which additional heterologous nucleic acid encodes at least one of β-catenin, CBP, TCF, and LEF.

24. The method of claim 22, wherein the cell is an embryonic stem cell or a neural stem cell cultured in the presence of retinoic acid (RA) and forskolin (FSK).
